SEQUENCE LISTING (6 sequences)

SEQ ID NO: 1 - DNA, 21 bp, Artificial
  randomly generated nucleotide sequence
tagtcacttcgatcgctgagg

SEQ ID NO: 2 - DNA, 20 bp, Artificial
  randomly generated nucleotide sequence
ggctcggcctctgagctaat

SEQ ID NO: 3 - DNA, 36 bp, Escherichia coli
tcgggccccaaataatgattttattttgactgatag

SEQ ID NO: 4 - DNA, 45 bp, Escherichia coli
atgggcccaaataatgattttattttgactgatagtgacctgttc

SEQ ID NO: 5 - DNA, 25 bp, Artifical sequence
  Made in the lab
tcgtcagtcgcagtctcagactcac

SEQ ID NO: 6 - DNA, 25 bp, Artifical sequence
  Made in the lab
gcugaugcgaugaaugaacacgaaa

(19) United States
(12) Patent Application Publication
(10) Pub. No.: US 2011/0071047 A1
(43) Pub. Date: Mar. 24, 2011

(54) PROMOTER DETECTION AND ANALYSIS

(75) Inventors: Yongsheng MA, Boise, ID (US); Xavier DANTHINNE, Boise, ID (US)

(73) Assignee: O.D. 260 INC., Boise, ID (US)

(21) Appl. No.: 12/890,350

(22) Filed: Sep. 24, 2010

Related U.S. Application Data
(63) Continuation-in-part of application No. 11/925,837, filed on Oct. 27, 2007.

(51) Int. Cl. C40B 30/04; C12Q 1/68; C12N 15/63; C40B 40/06

(52) U.S. Cl. 506/9; 435/6; 435/320.1; 506/16

(57) ABSTRACT

An array-based method for promoter detection and analysis is provided. Promoter sequence candidates are analyzed simultaneously in one reaction vial utilizing a plurality of vectors, each comprising a unique TAG sequence wherein transcriptional products are tagged as they are synthesized, in such a way that one specific transcript is labeled with only one type of tag, and one tag labels only one type of transcript. The transcriptional output is analyzed on conventional arrays or by real-time RT PCR.

### REFERENCE TO RELATED APPLICATIONS

This application is a continuation-in-part of U.S. patent application Ser. No. 11/925,837, filed Oct. 27, 2007.

This invention was made with government support under Grant 1R43HG003559 awarded by the National Institutes of Health. The government has certain rights in the invention.

### TECHNICAL FIELD

The present disclosure relates to methods for detecting regulatory elements in a cell sample. More specifically, the disclosure relates to methods for detecting regulatory elements in multiple cell samples at the same time and uses arising there from. The present disclosure also provides vectors for detection and analysis of regulatory elements.

### BACKGROUND

The genes of all living organisms are encoded by the nucleic acids DNA and RNA. Each gene encodes a protein that may be produced by the organism through expression of the gene.

The systems that regulate gene expression respond to a wide variety of developmental and environmental stimuli, thus allowing each cell type to express a unique and characteristic subset of its genes, and to adjust the dosage of particular gene products as needed. The importance of dosage control is underscored by the fact that targeted disruption of key regulatory molecules in mice often results in drastic phenotypic abnormalities (Johnson, R. S., et al., Cell, 71:577-586 (1992)), just as inherited or acquired defects in the function of genetic regulatory mechanisms contribute broadly to human disease.

Standard molecular biology techniques have been used to analyze the expression of genes in a cell by measuring nucleic acids. These techniques include PCR, northern blot analysis and other types of DNA probe analysis, such as in situ hybridization. Each of these methods allows one to analyze the transcription of only known genes and/or small numbers of genes at a time (Nucl. Acids Res. 19, 7097-7104 (1991); Nucl. Acids Res. 18, 4833-4842 (1990); Nucl. Acids Res. 18, 2789-2792 (1989); European J. Neuroscience 2, 1063-1073 (1990); Analytical Biochem. 187, 364-373 (1990); Genet. Annal Techn. Appl. 7, 64-70 (1990); GATA 8(4), 129-133 (1991); Proc. Natl. Acad. Sci. USA 85, 1696-1700 (1988); Nucl. Acids Res. 19, 1954 (1991); Proc. Natl. Acad. Sci. USA 88, 1943-1947 (1991); Nucl. Acids Res. 19, 6123-6127 (1991); Proc. Natl. Acad. Sci. USA 85, 5738-5742 (1988); Nucl. Acids Res. 16, 10937 (1988)).

Measurement of the levels of mRNA has also been used to monitor gene expression. Since proteins are transcribed from mRNA, it is possible to detect transcription by measuring the amount of mRNA present. One common method, called “hybridization subtraction”, allows one to look for changes in gene expression by detecting changes in mRNA expression (Nucl. Acids Res. 19, 7097-7104 (1991); Nucl. Acids Res. 18, 4833-4842 (1990); Nucl. Acids Res. 18, 2789-2792 (1989); European J. Neuroscience 2, 1063-1073 (1990); Analytical Biochem. 187, 364-373 (1990); Genet. Annal Techn. Appl. 7, 64-70 (1990); GATA 8(4), 129-133 (1991); Proc. Natl. Acad. Sci. USA 85, 1696-1700 (1988); Nucl. Acids Res. 19, 1954 (1991); Proc. Natl. Acad. Sci. USA 88, 1943-1947 (1991); Nucl. Acids Res. 19, 6123-6127 (1991); Proc. Natl. Acad. Sci. USA 85, 5738-5742 (1988); Nucl. Acids Res. 16, 10937 (1988)).

Gene expression has also been monitored by measuring levels of the gene product, (i.e., the expressed protein), in a cell, tissue, organ system or even organism. Measurement of gene expression by measuring the protein gene product may be performed using antibodies known to bind to the particular protein to be detected. A difficulty arises in needing to generate antibodies to each protein to be detected. Measurement of gene expression via protein detection may also be performed using two-dimensional gel electrophoresis, wherein proteins can, in principle, be identified and quantified as individual bands, and ultimately reduced to a discrete signal. In order to positively analyze each band, each band must be excised from the membrane and subjected to protein sequence analysis (e.g., Edman degradation). However, it can be difficult to isolate a sufficient amount of protein to obtain a reliable protein sequence. In addition, many of the bands often contain multiple proteins.

Another difficulty associated with quantifying gene expression by measuring the amount of protein gene product in a cell is that protein expression is an indirect measure of gene expression. It is impossible to know from measuring the amount of a protein present in a cell when expression of that protein occurred. Thus, it is difficult to determine whether the protein expression changes over time due to cells being exposed to different stimuli.

Measurement of the amount of particular activated transcription factors has been used to monitor gene expression. Transcription in a cell is controlled by activated transcription factors which bind to DNA at sites outside the core promoter for the gene and activate transcription. Detection of the presence of activated transcription factors is thus useful for measuring gene expression. Transcriptional activators are found in prokaryotes, viruses and eukaryotes.

A reporter gene (often simply referred to as a reporter) is a gene that researchers often attach to another gene of interest in cell culture, animals or plants. Certain genes are chosen as reporters because the characteristics they confer on organisms expressing them are easily identified and measured, or because they are selectable markers. Reporter genes are generally used to determine whether the gene of interest has been taken up by, or expressed in, the cell or organism population.

To introduce a reporter gene into an organism, researchers place the reporter gene and the gene of interest in the same DNA construct to be inserted into the cell or organism. For bacteria or eukaryotic cells in culture, this is usually in the form of a circular DNA molecule called a plasmid. It is important to use a reporter gene that is not natively expressed in the cell or organism under study, since the expression of the reporter is used as a marker for successful uptake of the gene of interest.

Commonly used reporter genes that induce visually identifiable characteristics usually involve fluorescent proteins; for example, green fluorescent protein (GFP) and the luciferase assay. Other reporters include, for example, beta-galactosidase, X-gal and chloramphenicol acetyltransferase (CAT).

Many methods of transfection and transformation—two ways of expressing a foreign or modified gene in an organism—are effective in only a small percentage of a population subjected to the techniques. Thus, a method for identifying those few successful gene uptake events is necessary. Reporter genes used in this way are normally expressed under their own promoter independent from that of the introduced gene of interest; the reporter gene can be expressed constitutively (“always on”) or inducibly with an external intervention such as the introduction of IPTG in the beta-galactosidase system. As a result, the reporter gene's expression is independent of the gene of interest's expression, which is an advantage when the gene of interest is only expressed under certain specific conditions or in tissues that are difficult to access.

In the case of selectable-marker reporters such as CAT, the transfected population of bacteria can be grown on a substrate that contains chloramphenicol. Only those cells that have successfully taken up the construct containing the CAT gene will survive and multiply under these conditions.

Reporter genes can also be used to assay for the expression of the gene of interest, which may produce a protein that has little obvious or immediate effect on the cell culture or organism. In these cases the reporter is directly attached to the gene of interest to create a gene fusion. The two genes are under the same promoter and are transcribed into a single polypeptide chain. In these cases it is important that both proteins be able to properly fold into their active conformations and interact with their substrates despite being fused. In building the DNA construct, a segment of DNA coding for a flexible polypeptide linker region is usually included so that the reporter and the gene of interest will only minimally interfere with one another.

Reporter genes can be used to assay for the activity of a particular promoter in a cell or organism. In this case there is no separate “gene of interest”; the reporter gene is simply placed under the control of the target promoter and the reporter gene product's activity is quantitatively measured. The results are normally reported relative to the activity under a “consensus” promoter known to induce strong gene expression.

In the past few years, the sequencing of numerous genomes, both eukaryotic and prokaryotic, has generated an enormous amount of data. Although detection of coding regions is common, the major challenge is to annotate the functional non-coding sequences, in particular those involved in gene transcription. Because transcription plays a pivotal role in regulating important processes such as morphogenesis, cell differentiation, tissue specificity, hormonal communication, and cellular stress responses, a need for the identification and functional characterization of transcriptional promoters exists. The methods for detection and analysis of transcriptional promoters can be divided into two categories: computational methods and experimental methods.

Computational methods for promoter studies incorporate the many public and private databases containing information gathered from studies published by hundreds of laboratories and conducted using conventional labor-intensive and time-consuming approaches. The Eukaryotic Promoter Database (EPD) and the Transcription Regulatory Regions Database (TRRD) contain 1,871 and 703 entries of human promoters, respectively. Other promoter databases, such as TransFac and DBTSS, contain almost 9,000 promoter sequences. However, most of these are derived from in silico primer extension assays (e.g., TransFac), or contain only data about the putative transcriptional start site (e.g., DBTSS). The small numbers of experimentally validated human promoters compared to the 35,000 expected human genes indicate the magnitude of the work still to be done.

Numerous computer-based promoter prediction methods have been developed (Scherf et al., J. Mol. Biol. 297(3):599-606, 2000; Werner, T. Brief Bioinform. 1(4):372-80, 2000; Loots et al., Gen. Res. 12:832-839, 2002). These methods are limited by the lack of a reliable, standard protocol to predict and identify promoter regions. Promoters are generally only a few base pairs (bp) long, and are embedded within the massive genome. Thus, promoters are much more difficult to find and are easier to confuse than long, patterned coding sequences. Typical computer algorithms for promoter prediction are based on comparisons of unknown sequences with known elements, a strategy which does not allow for identification of new types of promoter elements. Thus, computer-based searches for promoter elements are incomplete and always require experimental confirmation.

Computational methods based on microarray data have been used to investigate genome-wide transcriptional regulation (Pilpel et al., Nat. Gen. 29(2):153-9, 2001). These techniques allow for the identification of novel functional motif combinations in the promoters of a given organism, and may provide a global view of transcription networks. However, the data provided from these methods also need confirmation by experimental means.

The experimental methods for investigation of a promoter region and subsequent characterization usually follow a basic protocol. First, upon identification of a new coding sequence, the transcription start site is defined with standard molecular biology tools such as S1 mapping, primer extension, or 5′RACE. Second, the upstream genomic region (up to 10 kb) is cloned and demonstrated to have promoter activity by performing a reporter assay in a transient transfection system. Third, deletion and point mutation analyses are performed to define the important transcriptional cis-acting elements; information about transcriptional regulation may be obtained by applying different induction or repression agents in transient transfection assays. Finally, the transcription factors involved in promoter regulation are identified by Dnase I footprinting, electrophoresis mobility shift assay (EMSA) in the presence or absence of mutant probes and competitors, and EMSA supershift assay.

Transient-transfection based experimental methods have several disadvantages. These methods measure reporter protein level instead of mRNA level, which is the direct product of the transcription; protein levels may not always correlate with mRNA levels. There are a limited number of reporter assays available (e.g. chloramphenicol acetyl-transferase, β-galactosidase, luciferase, green fluorescent protein (GFP), β-glucuronidase) and the utilization of the same reporter to compare various promoters implies that these promoters must be tested separately and thus these assays are labor-intensive and time-consuming. Since each of the many steps involved (i.e. transfection, induction, harvest, reporter detection) are performed separately for each promoter investigated, usually in duplicate or triplicate, the handling of more than 20 constructs simultaneously is challenging. For each step performed, the time difference between the first and last sample may be significant; therefore incubation periods, cell and reagent quality, for example, may differ from one sample to the other thus introducing more experimental variation. Large amounts of material and reagents are required. Additionally, in order to compare a series of promoters to each other, a second reporter cassette has to be included as an internal control. In some instances, the detection of this control may be as time-consuming and labor-intensive as for the first reporter, and subject to experimental errors. The expression of this internal control can also compete with the gene expression driven by the promoter of interest, and affect the results of the assay. Some assays, such as luciferase and GFP assays, require expensive instrumentation.

Kim et al. reported an experimental method for isolation and identification of promoters in the human genome (Kim et al. Genome Research 15:830-839, 2005). However, the use of antibodies to identify regions that may be associated with active transcription and the required binding of both RNAP and TFIID as criteria for promoters may lead to the elimination of some promoters that only show partial binding.

Khambata-Ford et al. reported an experimental method for identification of promoter regions in the human genome by using a retroviral plasmid library-based functional reporter gene assay (Khambata-Ford et al., Gen. Res. 13:1765-1774, 2003). However, in addition to allowing potentially lethal disruption of the target cell genome by random integration of the retroviral vector, the assay relies on the fluorescent reporter GFP for detection and screens the cells via fluorescence-activated cell sorting (FACS).

Trinklein et al, reported an experimental method for identification and functional analysis of human transcriptional promoters (Trinklein et al., Gen. Res. 13:308-312, 2003) by using a draft sequence of the human genome and cDNA libraries. However, for further analysis and identification of promoter sequences they used a luciferase-based transfection assay.

The sequencing of genomes has generated a huge amount of data that needs to be annotated. Computational methods are available to detect putative transcriptional promoter regions, but they are not 100% efficient and must be confirmed by experimentation. The experimental procedures that are currently available to study promoters are time-consuming, laborious and not easily adapted to large numbers of promoters. Therefore, new techniques for transcriptional studies are needed.

### SUMMARY

The foregoing disadvantages of the previously described methods are overcome by providing a novel reporter system that incorporates unique, non-coding DNA sequences, and that is specific, inexpensive and provides an efficient means of promoter detection.

The present disclosure provides a method for the detection and analysis of DNA promoter sequences. In one embodiment, the present disclosure provides a method for detecting DNA regulatory sequences comprising: a) inserting each of a plurality of DNA regulatory sequence candidates or promoter sequence candidates into one of a plurality of vectors wherein each of the vectors comprises a unique TAG sequence and wherein each DNA regulatory sequence candidate or promoter sequence candidate is inserted in a position to drive transcription of the TAG sequence; b) inserting each of the vectors into one of a plurality of cloning host cells; c) growing the cloning host cells to the same optical density, pooling the cloning host cells, and extracting and purifying the vectors and inserting the vectors into a reporter cell line or a nuclear extract thereof; and d) extracting mRNA from the reporter cell line or nuclear extract and analyzing the mRNA. In certain embodiments, the mRNA extracted from the reporter cell line is directly labeled or is used as template for cDNA or probe synthesis, and the labeled mRNA, cDNA or probe is analyzed with an array wherein the array comprises identical or complementary sequence to the TAG sequences. Preferably, the labeled mRNA, cDNA or probe hybridizes to the array and the label of the mRNA, cDNA or probe has a detectable response.

In a further embodiment, a method for detecting DNA regulatory sequences or promoter sequences is provided, the method comprising: (a) inserting each of a plurality of DNA regulatory sequence candidates or promoter sequence candidates into one of a plurality of vectors wherein each of the vectors comprises a unique TAG sequence and wherein each DNA regulatory sequence candidate or promoter sequence candidate is inserted in a position to drive transcription of the TAG sequence; (b) inserting each of the vectors into one of a plurality of cloning host cells; (c) growing the cloning host cells to the same optical density; (d) extracting and purifying the vectors from the cloning host cells; (e) pooling the purified vectors and inserting the vectors into a reporter cell line; and (f) extracting mRNA from the reporter cell line and analyzing the mRNA, wherein the presence of mRNA corresponding to the TAG sequence from a specific vector is indicative of the presence of a DNA regulatory sequence or promoter sequence in the vector.

In certain embodiments, the length of the unique TAG sequences employed in the disclosed methods is between about 16 base pairs and about 200 base pairs, such as between about 20 base pairs and about 175 base pairs, between about 25 base pairs and about 150 base pairs, between about 30 base pairs and about 125 base pairs, between about 45 base pairs and about 100 base pairs, between about 50 base pairs and about 75 base pairs, or about 65, 60 or 21 base pairs. In another embodiment, all the TAG sequences are designed to have approximately the same melting temperature; this feature allows for the unbiased quantification of various mRNAs by hybridization under the same temperature and ionic strength conditions. In another embodiment, the method enables the detection and quantification of mRNA levels, instead of reporter protein levels, and is unaffected by potentially interfering translation and posttranslational events as in the conventional reporter assays.

In yet another embodiment, a method for detecting DNA regulatory sequences or promoter sequences is provided, the method comprising: (a) inserting each of a plurality of DNA regulatory sequence candidates or promoter sequence candidates into one of a plurality of vectors wherein each of the vectors comprises a unique TAG sequence and a labeled probe sequence, wherein each vector contains the same probe sequence and wherein each DNA regulatory sequence candidate or promoter sequence candidate is inserted in a position to drive transcription of the TAG sequence; (b) pooling equimolar amounts of the vectors; (c) introducing the vectors into reporter cells; (d) extracting and purifying RNA from the reporter cells; and (e) quantifying the amount of mRNA generated from each vector using real time reverse transcription polymerase chain reaction (real time RT-PCR), wherein the presence of mRNA corresponding to the TAG sequence from a specific vector is indicative of the presence of a DNA regulatory sequence or promoter sequence in the vector. The real time RT-PCR employs a forward primer that matches the sequence of a RNA adaptor ligated to the 5′ end of every intact RNA and reverse primers that are specific for the unique TAG sequences. The determined quantity of each type of RNA provides a measure of the transcriptional activity of each of the DNA regulatory or promoter candidate sequences.

In another embodiment, the present disclosure provides a method for the detection and analysis of DNA regulatory sequence candidates or promoter sequence candidates wherein the DNA regulatory sequence candidates or promoter sequence candidates are each integrated into one of a plurality of vectors that each comprise: a unique TAG sequence; one or more multiple-cloning sites; one or more DNA recombination sequences; a negative selection marker; one or more nucleotide sequences useful for the detection of mRNA sequences, such as a T7 promoter sequence and/or a MA segment; a translation stop codon; a RNA stabilization fragment, such as the one from the alpha-globin gene; and a transcription termination signal, such as a poly A signal, and wherein the DNA regulatory sequence candidates or promoter sequence candidates are located such that they drive the transcription of the TAG sequences.

In another embodiment, the present disclosure provides a method for the detection and analysis of DNA regulatory sequence candidates or promoter sequences wherein the DNA regulatory sequence candidates or promoter sequence candidates are each integrated into one of a plurality of vectors that each comprise a unique TAG sequence, one or more multiple-cloning sites, both of attP1 and attP2 sequences, a negative selection marker wherein the negative selection marker is the ccdB gene, a T7 promoter sequence, a MA segment, a translation stop codon, an alpha-globin RNA stabilization fragment, and a poly A-signal, wherein the DNA regulatory sequence candidates or promoter sequence candidate drives the transcription of the TAG sequence.

In another embodiment, the present disclosure provides a method for the detection and analysis of DNA regulatory sequence candidates or promoter sequences wherein DNA regulatory sequence candidates or promoter sequence candidates are each integrated into one of a plurality of vectors wherein each of the vectors comprises a unique TAG sequence, one or more multiple-cloning sites, both of attP1 and attP2 sequences, a negative selection marker, a T7 promoter sequence, a MA sequence wherein the MA sequence is comprised of approximately 25% A, 25% T, 25% G and 25% C, a translation stop codon, a RNA stabilization fragment, and a transcription termination signal, wherein the DNA regulatory sequence candidates or promoter sequence candidates drive the transcription of the unique TAG sequences. Preferably, the vector is a plasmid. Preferably, the RNA stabilization fragment is from an alpha-globin gene. Preferably, the transcription termination signal is a poly A signal.

In another embodiment, the present disclosure provides a method for the detection and analysis of DNA regulatory sequence candidates or promoter sequences wherein DNA regulatory sequence candidates or promoter sequence candidates are each integrated into one of a plurality of vectors wherein each of the vector comprises a unique TAG sequence, one or more multiple-cloning sites, one or more DNA recombination sequences, a negative selection marker, a T7 promoter sequence, a MA sequence wherein the MA sequence is comprised of approximately 25% A, 25% T, 25% G, and 25% C, a translation stop codon wherein the translation stop is in three frames, a RNA stabilization fragment, and a transcription termination signal, and wherein the DNA regulatory sequence candidate or promoter sequence candidate is located such that it drives the transcription of the TAG sequence. Preferably, the vector is a plasmid. Preferably, the RNA stabilization fragment is from an alpha-globin gene. Preferably, the transcription termination signal is a poly A signal. Preferably, the DNA recombination sequences are attP1 and attP2.

In another embodiment, the present disclosure provides a method for the detection and analysis of DNA promoter sequences comprising: (a) integrating DNA regulatory sequence candidates or promoter sequence candidates within TAG-vectors, wherein the DNA regulatory sequence candidate or promoter sequence candidate is located such that it drives the transcription of a unique TAG sequence, wherein the TAG-vector comprises: at least one multiple cloning site (MCS) for inserting the DNA regulatory sequence candidate or promoter sequence candidate; DNA recombination sequences, such as attP1 and attP2, between which DNA regulatory sequence candidates or promoter sequence candidates can be inserted; a negative selection marker to maximize the recovery of clones containing regulatory sequence or promoter sequence inserts, such as ccdB; a nucleotide sequence useful to enable RNA synthesis, preferably a T7 promoter sequence; a unique reporter TAG, a specific MA segment useful to synthesize probes from RNA, wherein the MA segment is comprised of approximately 25% A, 25% T, 25% G, and 25% C; a three frame translation stop codon; RNA stabilization fragment, preferably from a hemoglobin or alpha-globin gene; and a transcription termination signal, such as a poly A-signal; (b) cloning the TAG-vectors with the regulatory sequence candidate or promoter sequence candidate inserts into a host, preferably Escherichia coli, arraying the clones are into a 96-well plate, and growing the clones to about the same cell density; (c) pooling the resultant clones, and purifying the vectors; (d) transfecting the purified vector mixture into a cell line of interest; and (e) extracting, labeling and quantifying the RNA by hybridization to the DNA TAG sequences arrayed on a support, such as a membrane, glass support or beads. Suitable bead compositions include those used in peptide, nucleic acid and organic moiety synthesis, such as but not limited to, plastics, ceramics, glass, polystyrene, methylstyrene, acrylic polymers, paramagnetic materials, thoria sol, carbon graphite, titanium dioxide, latex or cross-linked dextrans such as sepharose, cellulose, nylon, cross-linked micelles and Teflon™ (see Microsphere Detection Guide, Bangs Laboratories, Fishers Ind.). Preferably, the vector is a plasmid. Preferably, the label of the mRNA, cDNA or probe has a detectable response.

In yet another embodiment, a method is provided wherein each DNA regulatory sequence candidate or promoter sequence candidate under investigation (for example, computer-predicted DNA promoter sequence candidates, DNA fragments from a collection of nucleotide sequences, such as a genomic library, deletion or site-directed mutants of a specific DNA promoter, tissue-specific promoters, artificial promoters, etc.) drives the transcription of a unique mRNA that consists of a unique short oligonucleotide TAG inserted upstream of the 5′ end of a luciferase coding sequence, wherein equimolar amounts of the various regulatory sequences or promoters under investigation are pooled and transfected into a cell line, and wherein the mRNA levels are quantified by hybridization to the TAG oligonucleotides in an array format. In another embodiment, the reporters are short oligonucleotides TAGs.

In certain embodiments of the present disclosure, each of the clones containing a TAG vector, preferably a plasmid, is grown to about the same cell density, the purified vectors, preferably plasmids, of these clonal cultures, containing every DNA regulatory sequence candidate or promoter sequence candidate, are mixed, and the resulting mixture is transfected into a single population of cells creating a competitive environment for the various promoters to recruit transcription factors. In another embodiment, vectors, preferably plasmids, purified from the clonal cell cultures of about equal cell density and containing about equimolar amounts of all the DNA regulatory sequences or promoter sequences are mixed and used for transfection of a single population of cells and the need for internal controls is eliminated. There are several ways to obtain equimolar amounts of the vectors that carry the various candidate sequences-TAG combinations that are used to transfect reporter cell lines. In one embodiment, equimolar amounts of the vectors are obtained by: 1) making the vector library; 2) arraying the vector library (e.g., in a 96 well plate); 3) taking an equal fraction from each clone and pooling them all; 4) growing all clones together assuming same growth rate and yield of the same amount of vector per cell; 5) extracting the transformation agent (e.g., vector, plasmid or virus); and 6) transfecting the vector (or plasmid or infect virus) into a reporter cell line. Alternately, equimolar amounts of the vectors are obtained by: 1) making the vector library; 2) arraying the vector library (e.g., in a 96 well plate); 3) growing each clone individually (e.g., in a deep-well plate in case of bacteria); 4) taking an equal fraction from each clone and pooling them all; 5) extracting the transformation agent (e.g., vector, plasmid or virus); and 6) transfecting the vector (or plasmid or infect virus) into the reporter cell line. In another embodiment, equimolar amounts of the vectors are obtained by: 1) making the vector library; 2) arraying the vector library (e.g., in a 96 well plate); 3) growing each clone individually (e.g., in a deep-well plate in case of bacteria); 4) extracting the transformation agent (e.g., vector, plasmid or virus) and quantifying it; 5) taking an equal fraction from each clone (e.g., vector, plasmid or virus) and pooling them all; and 6) transfecting vector (or plasmid or infect virus) into the reporter cell line. In yet a further embodiment, equimolar amounts of the vectors are obtained by: 1) making the vector library; 2) taking a fraction from each clone, and pooling them all; 3) growing all the clones together and assuming the same growth rate and yield of the same amount of vector per cell; 4) extracting transformation agent (e.g., vector, or plasmid or virus); 5) transfecting vector (or plasmid or infect virus) into a reporter cell line and identifying the TAG of interest (e.g., by high level of expression); and 6) finding the clone in the vector library that contains the TAG of interest (e.g., by colony hybridization).

In another embodiment, the present disclosure provides a method for the detection and analysis of DNA regulatory sequences or promoter sequences comprising: (a) integrating each DNA regulatory sequence or promoter sequence candidates into one of a plurality of vectors wherein each vector comprises a unique TAG sequence, one or more multiple-cloning sites, at least one DNA recombination sequence, preferably attP1 or attP2, a negative selection marker, preferably ccdB, a nucleotide sequence useful to enable RNA synthesis, such as a T7 promoter sequence, a MA segment, a translation stop codon, a RNA stabilization fragment, preferably from the hemoglobin or alpha-globin gene, and transcription termination signal, such as a poly A-signal, wherein the DNA promoter sequence candidate is located such that it drives the transcription of the TAG sequence; (b) cloning the vectors with the promoter sequence candidate inserts into a host, preferably Escherichia coli, arraying the clones into a 96-well plate and growing the cells to the same cell density; (c) pooling the resultant clones, and purifying the vectors therein; (d) transfecting the purified vector mixture into a cell line of interest whereby the use of internal controls is eliminated and (e) extracting the RNA. The RNA can be directly labeled or used as a template for cDNA or probe synthesis and then quantified by hybridization to the TAG sequences arrayed on a membrane or glass support. Preferably, the vector is a plasmid. Preferably, the label of the mRNA, cDNA or probe has a detectable response.

In another embodiment, the disclosure provides a method for the detection and analysis of DNA regulatory sequences or promoter sequences comprising integrating DNA regulatory sequence or promoter sequence candidates into one of a plurality of vectors, preferably a plasmid, wherein each vector comprises a unique TAG sequence, one or more multiple-cloning sites, at least one DNA recombination sequence, preferably attP1 or attP2, a negative selection marker, such as ccdB, a nucleotide sequence useful to enable RNA synthesis, preferably a T7 promoter sequence, a MA segment, a translation stop codon, an RNA stabilization fragment, preferably a hemoglobin or alpha-globin gene, and transcription termination signal, preferably a poly A-signal, and wherein the DNA promoter sequence candidate is located such that it drives the transcription of the TAG sequence.

In another embodiment, the present disclosure provides a method for the detection and analysis of DNA regulatory sequences or promoter sequences comprising: (a) integrating each DNA regulatory sequence or promoter sequence candidate into one of a plurality of vectors wherein each vector comprises a unique TAG sequence, one or more multiple-cloning sites, at least one DNA recombination sequence, a negative selection marker, a nucleotide sequence useful to enable RNA synthesis, a MA segment, a translation stop codon, a RNA stabilization fragment, and a transcription termination signal, wherein the DNA regulatory sequence or promoter sequence candidate is located such that it drives the transcription of the TAG sequence; (b) cloning the vectors with the regulatory sequence or promoter sequence candidate inserts into a host, preferably Escherichia coli, and arraying the clones into a 96-well plate and growing to the same cell density; (c) pooling the resultant clones, and purifying the vectors therein; (d) transfecting the purified vector mixture into a cell line of interest; and (e) extracting, labeling and quantifying the RNA by hybridization to the DNA TAG sequences arrayed on a support such as a membrane or glass support. Preferably, the vector is a plasmid. Preferably, the DNA recombination sequence is attP1 or attP2. Preferably, the nucleotide sequence useful to enable RNA synthesis is a T7 promoter sequence. Preferably, the transcription termination signal is a poly A-signal. Preferably, the RNA stabilization fragment is from the hemoglobin or alpha-globin gene. Preferably, the label of the mRNA, cDNA or probe has a detectable response.

In another embodiment, the present disclosure provides a method for the detection and analysis of DNA regulatory sequences or promoter sequences comprising: (a) integrating each of the DNA regulatory sequence or promoter sequence candidates into one of a plurality of vectors wherein each vector comprises a unique TAG sequence, one or more multiple-cloning sites, at least one DNA recombination sequence, a negative selection marker, a nucleotide sequence useful to enable RNA synthesis, a MA segment, a translation stop codon, a RNA stabilization fragment, and a transcription termination signal, wherein the DNA promoter sequence candidate is located such that it drives the transcription of the TAG sequence; (b) cloning the vectors with the regulatory sequence or promoter sequence candidate inserts into a host, preferably Escherichia coli, arraying the clones into a 96-well plate and growing the clones to the same cell density; (c) pooling the resultant clones, and purifying the vectors therein; (d) transfecting the purified vector mixture into a cell line of interest, whereby the use of internal controls is eliminated upon transfecting the cells with vectors purified from the clonal cell populations which are of the same cell density; and (e) extracting, labeling and quantifying the RNA by hybridization to the DNA TAG sequences arrayed on a membrane or glass support. Preferably, the vector is a plasmid. Preferably, the DNA recombination sequence is attP1 or attP2. Preferably, the nucleotide sequence useful to enable RNA synthesis is a T7 promoter sequence. Preferably, the RNA stabilization fragment is from the hemoglobin or alpha-globin gene. Preferably, the transcription termination signal is a poly A-signal. Preferably, the label of the mRNA, cDNA or probe has a detectable response.

In another embodiment of the present disclosure, the disclosure provides a method for detection and analysis of DNA regulatory or promoter nucleotide sequences in a collection of nucleotide sequences, such as genomic library, comprising: (a) mixing regulatory or promoter sequence candidates with TAG-vectors, wherein each TAG-vector comprises: at least one multiple cloning site (MCS) for inserting promoter sequence candidate, at least one DNA recombination sequence, such as attP1 or attP2, a negative selection marker to maximize the recovery of clones containing promoter sequence inserts, such as, for example, a ccdB gene, a T7 promoter sequence to enable RNA synthesis, a unique approximate 60 base pair reporter TAG, a specific MA segment useful to synthesize probes from RNA, wherein the MA segment is comprised of approximately 25% A, 25% T, 25% G, and 25% C, a three frame translation stop codon, a RNA stabilization fragment, such as, for example, alpha-globin or hemoglobin, and transcription termination signal, preferably a poly A-signal; (b) cloning the TAG-vectors with the promoter sequence candidate inserts into a host, preferably Escherichia coli, arraying the clones into a 96-well plate and growing the clones to the same cell density; (c) pooling the resultant clones, and purifying the vectors therein; (d) transfecting the purified vector mixture into a cell line of interest; and (e) extracting, labeling and quantifying the RNA by hybridization to the DNA TAG sequences arrayed on a membrane or glass support. Preferably, the TAG-vector is a TAG-plasmid. Preferably, the label of the mRNA, cDNA or probe has a detectable response.

In another embodiment of the present disclosure, the disclosure provides a method for the detection and analysis of DNA regulatory or promoter nucleotide sequences in a collection of nucleotide sequences, such as a genomic library, comprising: (a) mixing regulatory or promoter sequence candidates with TAG-vectors, wherein each TAG-vector comprises: at least one multiple cloning site (MCS) for inserting the regulatory or promoter sequence candidate, at least one DNA recombination sequence, a negative selection marker, a nucleotide sequence useful to enable RNA synthesis, a unique approximate 60 base pair reporter TAG, a specific MA segment useful to synthesize probes from RNA, wherein the MA segment is comprised of approximately 25% A, 25% T, 25% G, and 25% C, a three frame translation stop codon, a RNA stabilization fragment, and transcription termination signal; (b) cloning the TAG-vectors with the regulatory or promoter sequence candidate inserts into a host, preferably Escherichia coli, arraying the clones into a 96-well plate and growing the clones to the same cell density; (c) pooling the resultant clones, and purifying the vectors therein; (d) transfecting the purified vectors into a cell line of interest without the use of internal controls; and (e) extracting, labeling and quantifying the RNA by hybridization to the DNA TAG sequences arrayed on a membrane or glass support. Preferably, the vectors are plasmids. Preferably, the DNA recombination sequence is attP1 or attP2. Preferably, the negative selection marker is ccdB. Preferably, the nucleotide sequence to enable RNA synthesis is a T7 promoter sequence. Preferably, the RNA stabilization fragment is from the alpha-globin gene. Preferably, the transcription termination signal is a poly A-signal. Preferably, the label of the mRNA, cDNA or probe has a detectable response.

In another embodiment, the disclosure provides a method for detection and analysis of DNA regulatory or promoter nucleotide sequences in a collection of nucleotide sequences, such as a genomic library, comprising: (a) mixing regulatory or promoter sequence candidates with TAG-vectors, wherein each TAG-vector comprises: at least one multiple cloning sites (MCS) for inserting the regulatory or promoter sequence candidate, at least one DNA recombination sequence, a negative selection marker, a nucleotide sequence useful to enable RNA synthesis, a unique approximate 60 base pair reporter TAG, a specific MA segment useful to synthesize probes from RNA, wherein the MA segment is comprised of approximately 25% A, 25% T, 25% G, and 25% C, a three frame translation stop codon, a RNA stabilization fragment, and a transcription termination signal; (b) cloning the TAG-vector with the promoter sequence candidate inserts into a host, preferably Escherichia coli, arraying the clones into a 96-well plate and growing the clones to the same cell density; (c) pooling the resultant clones, containing about equal amounts of vectors, and purifying the vectors therein; (d) transfecting the purified vectors into a cell line of interest without the use of internal controls; and (e) extracting, labeling and quantifying the RNA by hybridization to the DNA TAG sequences arrayed on a membrane or glass support. Preferably, the TAG-vectors are TAG-plasmids. Preferably, the DNA recombination sequence is attP1 or attP2. Preferably, the negative selection marker is ccdB. Preferably, the nucleotide sequence to enable RNA synthesis is a T7 promoter sequence. Preferably, the RNA stabilization fragment is from the alpha-globin gene. Preferably, the transcription termination signal is a poly A-signal. Preferably, the label of the mRNA, cDNA or probe has a detectable response.

In another embodiment, the disclosure provides a method for analysis and detection of a plurality of DNA regulatory or promoter nucleotide sequences in a plurality of samples, comprising: (a) mixing DNA regulatory or promoter sequence candidates, wherein the DNA regulatory or promoter sequence candidates are, for example, selected from computer-predicted promoter sequence candidates, DNA fragments from a collection of nucleotide sequences, such as a genomic library, deletion or site-directed mutants of a specific promoter, tissue-specific promoters, artificial promoters, etc., with TAG vectors, wherein each TAG-vector comprises: at least one multiple cloning site for inserting the DNA regulatory or promoter sequence candidate, at least one DNA recombination sequence, a negative selection marker, a nucleotide sequence useful to enable RNA synthesis, a unique approximate 60 base pair reporter TAG, a specific MA segment useful to synthesize probes from RNA, wherein the MA segment is comprised of about 25% A, 25% T, 25% G, and 25% C, a three frame translation stop codon, a RNA stabilization fragment, and a transcription termination signal; (b) cloning the TAG-vectors with the promoter sequence candidate inserts into a host, such as Escherichia coli, arraying the clones into a 96-well plate and growing the clones to the same cell density; (c) pooling the resultant clones, and purifying the vectors therein; (d) transfecting the purified plasmid mixture into a cell line of interest; and (e) extracting, labeling and quantifying the RNA is extracted, labeled, by hybridization to the DNA TAG sequences arrayed on a membrane or glass support. Preferably, the TAG-vectors are TAG-plasmids. Preferably, the DNA recombination sequence is attP1 or attP2. Preferably, the negative selection marker is ccdB. Preferably, the nucleotide sequence to enable RNA synthesis is a T7 promoter sequence. Preferably, the RNA stabilization fragment is from the alpha-globin gene. Preferably, the transcription termination signal is a poly A-signal. Preferably, the label of the mRNA, cDNA or probe has a detectable response.

In another embodiment, the disclosure provides a method for detection and analysis of a plurality of DNA regulatory or promoter nucleotide sequences in a plurality of samples, comprising: (a) mixing DNA regulatory or promoter sequence candidates, wherein the regulatory or promoter sequence candidates are, for example, selected from computer-predicted promoter sequence candidates, DNA fragments from a collection of nucleotide sequences, such as a genomic library, deletion or site-directed mutants of a specific promoter, tissue-specific promoters, artificial promoters, etc., with TAG vectors, wherein each TAG-vector comprises: at least one multiple cloning site for inserting the regulatory or promoter sequence candidate, a DNA recombination sequence, a negative selection marker, a nucleotide sequence useful to enable RNA synthesis, a unique approximately 60 base pair reporter TAG, a specific MA segment useful to synthesize probes from RNA, wherein the MA segment is comprised of about 25% A, 25% T, 25% G, and 25% C, a three frame translation stop codon, a RNA stabilization fragment, and a transcription termination signal; (b) cloning the TAG-vectors with the regulatory or promoter sequence candidate inserts into a host, such as Escherichia coli, arraying the clones into a 96-well plate and growing the clones to the same cell density; (c) pooling the resultant clones containing about equal amounts of vector, and purifying the vectors therein; (d) transfecting about equal amounts of the purified vectors into a cell line of interest; and (e) extracting, labeling and quantifying the RNA by hybridization to the DNA TAG sequences arrayed on a membrane or glass support. Preferably, the TAG-vectors are TAG-plasmids. Preferably, the DNA recombination sequence is attP1 or attP2. Preferably, the negative selection marker is ccdB. Preferably, the nucleotide sequence to enable RNA synthesis is a T7 promoter sequence. Preferably, the RNA stabilization fragment is from the alpha-globin gene. Preferably, the transcription termination signal is a poly A-signal. Preferably, the label of the mRNA, cDNA or probe has a detectable response.

In another embodiment, the disclosure provides a method for the detection and analysis of a plurality of DNA regulatory or promoter nucleotide sequences in a plurality of samples, comprising: (a) mixing DNA regulatory or promoter sequence candidates, wherein the promoter sequence candidates are, for example, selected from computer-predicted promoter sequence candidates, DNA fragments from a collection of nucleotide sequences, such as a genomic library, deletion or site-directed mutants of a specific promoter, tissue-specific promoters, artificial promoters, etc., with a plurality of TAG vectors, wherein each TAG-vector comprises: at least one multiple cloning site for inserting promoter sequence candidate, a DNA recombination sequence, a negative selection marker, a nucleotide sequence useful to enable RNA synthesis, a unique approximate 60 base pair reporter TAG, a specific MA segment useful to synthesize probes from RNA, wherein the MA segment is comprised of about 25% A, 25% T, 25% G, and 25% C, a three frame translation stop codon, a RNA stabilization fragment, and a transcription termination signal; (b) cloning the TAG-vectors with the DNA regulatory or promoter sequence candidate inserts into a host, such as Escherichia coli, arraying the clones into a 96-well plate and growing the clones to the same cell density; (c) pooling the resultant clones, and purifying the vectors therein; (d) transfecting about equal amounts of the purified vectors into a cell line of interest without the use of internal controls; and (e) extracting, labeling and quantifying the RNA by hybridization to the DNA TAG sequences arrayed on a membrane or glass support. Preferably, the TAG-vectors are TAG-plasmids. Preferably, the DNA recombination sequence is attP1 or attP2. Preferably, the negative selection marker is ccdB. Preferably, the nucleotide sequence to enable RNA synthesis is a T7 promoter sequence. Preferably, the RNA stabilization fragment is from the alpha-globin gene. Preferably, the transcription termination signal is a poly A-signal. Preferably, the label of the mRNA, cDNA or probe has a detectable response.

In other embodiments, the present disclosure provides a plurality of vectors into which a plurality of DNA regulatory sequence or promoter sequence candidates can be inserted, wherein each of the vectors comprises a unique TAG sequence, at least one multiple cloning site and a transcription termination signal. In certain embodiments, the vectors further comprise at least one DNA recombination sequence, a negative selection marker, a RNA polymerase promoter sequence, a MA segment, a translation stop codon, and/or a RNA stabilization fragment. The DNA regulatory or promoter sequence candidate is inserted into the vector such that it can drive the transcription of the unique TAG sequence. Preferably, the vector is a plasmid. Kits are also provided by the present disclosure, such kits comprising a plurality of vectors disclosed herein and an array, wherein the array comprises sequences that are identical to, or complementary to, the unique TAG sequences.

In another embodiment, the present disclosure provides a plasmid vector comprising: a region for insertion of a putative regulatory sequence or promoter sequence wherein a multiple cloning site is located both 5′ and 3′ to the putative promoter or regulatory sequence; one or more DNA recombination sequences; a T7 sequence; a unique TAG sequence; a luciferase gene sequence; a MA sequence; and a translational stop sequence. Preferably, the MA sequence is either MA5 or MA4. Preferably, the MA sequence is located 3′ from the TAG sequence. Preferably, the luciferase gene sequence is partial luciferase gene sequence or the full luciferase gene sequence. Preferably, the translational stop sequence is a translational stop sequence in at least one reading frame, more preferably at least two reading frames, and most preferably in three reading frames. Preferably, the DNA recombination sequences are attP1 and attP2.

In another embodiment, the present disclosure provides a plasmid vector into which a DNA promoter or regulatory sequence is inserted, comprising a unique TAG sequence, one or more multiple-cloning sites, one or both of attP1 and attP2 sequences, a negative selection marker, a RNA polymerase promoter sequence, a MA segment, a translation stop codon, a RNA stabilization fragment, and a transcription termination signal, and wherein the DNA promoter or regulatory sequence is located such that it drives the transcription of the TAG sequence. Preferably, the vector is a plasmid. Preferably, the TAG sequence is between about 16 base pairs to about 200 base pairs, such as about 60 base pairs. Preferably, the TAG sequence is located 3′ to the inserted promoter sequence and 5′ to a transcription termination signal. Preferably, the DNA promoter sequence is an enhancer. Preferably, the translation stop codon is a three frame translation stop codon. Preferably, the RNA stabilization fragment is from an alpha-globin gene. Preferably, the transcription termination signal is a poly-A signal. Preferably, the RNA polymerase promoter sequence is a T7 promoter sequence.

In another embodiment, the disclosure provides a nucleotide sequence for use in the detection and analysis of a promoter or regulatory nucleotide sequence comprising: a T7 promoter, a unique TAG sequence, a MA sequence, and a poly A-signal. In certain embodiments, the promoter or regulatory sequence candidate is selected from promoter or regulatory sequence candidates provided by a computer-predicted model, DNA fragments from a collection of nucleotide sequences, such as a genomic library, deletion or site-directed mutants of a specific promoter, tissue-specific promoters, artificial promoters, etc. The unique TAG sequence is preferably a DNA sequence composed of random nucleotides. The length of the TAG sequence is short, such as between about 16 base pairs to about 200 base pairs, between about 20 base pairs to about 150 base pairs, between about 30 base pairs to about 120 base pairs, between about 40 base pairs to about 100 base pairs, between about 50 base pairs to about 75 base pairs, for example about 60 base pairs. Within a plurality of TAG sequences, each TAG sequence has approximately equivalent amounts of the nucleotides A, T, G, and C such that each TAG sequence has approximately the same melting temperature as the other TAGs, thereby allowing for the unbiased quantification of various mRNAs by hybridization under the same temperature and ionic strength conditions. In certain embodiments, the specific MA segment is useful to synthesize probes from RNA, and the MA segment is comprised of about 25% A, 25% T, 25% G, and 25% C.

In another embodiment, the disclosure provides a method in which a nucleotide sequence is used for the detection and analysis of a promoter or regulatory nucleotide sequence, the nucleotide sequence comprising: a T7 promoter sequence, a TAG sequence, a MA sequence, and a poly A-signal. A DNA promoter or regulatory sequence candidate may be selected from promoter or regulatory sequence candidates provided by a computer-predicted model, DNA fragments from a collection of nucleotide sequences, such as a genomic library, deletion or site-directed mutants of a specific promoter, tissue-specific promoters, artificial promoters, etc. In preferred embodiments, the TAG sequence is a DNA sequence comprised of short, random nucleotides, preferably between about 16 base pairs to about 200 base pairs, between about 20 base pairs to about 150 base pairs, between about 30 base pairs to about 120 base pairs, between about 40 base pairs to about 100 base pairs, and between about 50 base pairs to about 75 base pairs, for example about 60 base pairs in length.

In another embodiment, the present disclosure provides a cloning vector comprising a TAG sequence; a transcription termination signal, preferably a poly A-signal; a nucleotide sequence useful to enable RNA synthesis, preferably a T7 promoter sequence; and a MA sequence, wherein the nucleotide sequence useful to enable RNA synthesis and the MA sequence are on the antisense DNA strand. In another embodiment of the present disclosure, a cloning vector is provided wherein the cloning vector is comprised of a DNA promoter or regulatory sequence candidate; a TAG sequence; a transcription termination signal, such as a polyA signal; a nucleotide sequence useful to enable RNA synthesis, such as a T7 promoter sequence; and a MA sequence, wherein the DNA promoter sequence candidate, the TAG sequence, and the transcription termination signal, are located on the sense DNA strand.

In another embodiment of the present disclosure, a cloning vector is provided wherein the cloning vector is comprised of a TAG sequence; a transcription termination signal, such as poly A-signal; a nucleotide sequence useful to enable RNA synthesis, such as a T7 promoter sequence; and a MA sequence, wherein a DNA promoter sequence candidate is located 5′ to the TAG sequence and the TAG sequence is located 5′ to the transcription termination signal. In another embodiment of the present disclosure, a cloning vector is provided that comprises a TAG sequence; a transcription termination signal, such as a poly A-signal; a nucleotide sequence useful to enable RNA synthesis, such as a T7 promoter sequence; and a MA sequence, wherein the TAG sequence is located 3′ to a DNA promoter sequence candidate and the transcription termination signal, is located 3′ to the TAG sequence.

In another embodiment of the present disclosure, a cloning vector is provided that comprises: a TAG sequence; a transcription termination signal, such as a poly A-signal; a nucleotide sequence useful to enable RNA synthesis, such as a T7 promoter sequence; and a MA sequence, wherein a DNA promoter sequence is operably linked to the TAG sequence. In another embodiment of the present disclosure, a cloning vector is provided that comprises: a DNA promoter sequence candidate, a TAG sequence, a transcription termination signal, such as a poly A-signal; a nucleotide sequence useful to enable RNA synthesis, such as a T7 promoter sequence; and a MA sequence, wherein the TAG sequence is operably linked to the transcription termination signal.

In another embodiment of the present disclosure, a cloning vector is provided that comprises: a TAG sequence; a transcription termination signal, such as a poly A-signal; a nucleotide sequence useful to enable RNA synthesis, such as a T7 promoter sequence; and a MA sequence, wherein a DNA promoter sequence is located 5′ to the TAG sequence, the TAG sequence is located 5′ to the transcription termination signal, the transcription termination signal is located 3′ to a DNA promoter sequence candidate, the DNA promoter sequence candidate is operably linked to the TAG sequence and TAG sequence is operably linked to the transcription termination signal.

In another embodiment of the present disclosure, a cloning vector is provided wherein the cloning vector comprises: a pair of multiple cloning sites (MCS), a TAG sequence, a transcription termination signal, such as a poly A-signal, a nucleotide sequence useful to enable RNA synthesis, such as a T7 promoter sequence, and a MA sequence, wherein one MCS is located 5′ of a DNA promoter or regulatory sequence candidate and one MCS is located 3′ of the DNA promoter or regulatory sequence candidate.

The present disclosure provides an array-based method for promoter detection and analysis. The method provides for transcriptional products that are tagged as they are synthesized, in such a way that one specific transcript is labeled with only one type of TAG, and one TAG labels only one type of transcript. All promoter sequence candidates are analyzed simultaneously in one reaction vial. The transcriptional output is analyzed on conventional arrays and can be detected with procedures that do not require expensive instrumentation. The method fulfills the need for reduction of labor and costs, and provides for the detection of promoter regions from genomic libraries and other related advantages.

These and other embodiments of the present disclosure will become apparent upon reference to the detailed description and illustrative examples which are intended to exemplify non-limiting embodiments of the disclosure. All references disclosed herein are hereby incorporated by reference in their entirety as if each was incorporated individually.

### GLOSSARY

Unless defined otherwise, all technical and scientific terms used herein have the same meaning as commonly understood by one of ordinary skill in the art to which this disclosure belongs. Generally, the nomenclature used herein and the laboratory procedures in cell culture, molecular genetics, nucleic acid chemistry and hybridization described below are those well known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y., which is incorporated herein by reference) which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical and electronics terms as used herein are as defined in The New IEEE Standard Dictionary of Electrical and Electronics Terms (5.sup.th edition, 1993). As employed throughout the disclosure, the following terms, unless otherwise indicated, shall be understood to have the following meanings and are more fully defined by reference to the specification as a whole:

The term “amplified” refers to the construction of multiple copies of a nucleic acid sequence or multiple copies complementary to the nucleic acid sequence using at least one of the nucleic acid sequences as a template. Amplification systems include, for example, the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA) See, e.g., Diagnostic Molecular Microbiology: Principles and Applications, D. H. Persing et al., Ed., American Society for Microbiology, Washington, D.C. (1993). The product of amplification is termed an amplicon.

The term “array” refers to an array containing nucleic acid samples. An array may be a “macroarray” or a “microarray.” The term “microarray” refers to an array containing nucleic acid samples, also referred to as microscopic DNA ‘spots,’ bound to solid substrates, such as glass microscope slides, plastic, or silicon wafers. Because the physical area occupied by each sample is usually 50-200 μm in diameter, nucleic acid samples representing multiple samples, including, for example, entire genomes, genomic libraries, synthesized DNA samples from computer predicted models, or in deletion mutants of promoters under investigation etc., may be bound to the solid substrate. The solid substrate may include membranes or beads. Macroarrays may be such as those available commercially (Clontech) or synthesized manually. Beads may be of those used in peptide, nucleic acid and organic moiety synthesis, including but not limited to, plastics, ceramics, glass, polystyrene, methylstyrene, acrylic polymers, paramagnetic materials, thoria sol, carbon graphite, titanium dioxide, latex or cross-linked dextrans such as sepharose, cellulose, nylon, cross-linked micelles and Teflon™ many all be used (see Microsphere Detection Guide, Bangs Laboratories, Fishers Ind.). Microarrays allow the genes of a given sample to be simultaneously monitored with respect to some experimental condition of interest. Microarrays may be fabricated by the mechanical deposition of nucleic acid samples onto a solid substrate. Alternatively, the nucleic acid samples may be manually deposited. The term “DNA microarray” may apply to several different forms of the technology, each differing in the type of nucleic acid applied and the method of application.

The term “assay marker” or a “reporter gene” refers to a gene that can be detected, or ‘followed.’ The expression of the reporter gene may be measured at either the RNA level, or at the protein level. The gene product may be detected in experimental assay protocol, such as marker enzymes, antigens, amino acid sequence markers, cellular phenotypic markers, nucleic acid sequence markers, and the like. A “reporter gene” (or “reporter”) is a gene that researchers may attach to another gene of interest in cell culture, bacteria, animals, or plants. Some reporters are selectable markers, or confer specific characteristics upon on organisms expressing them, thereby allowing the organism to be easily identified and measured. To introduce a reporter gene into an organism, researchers place the reporter gene and the gene of interest in the same DNA construct to be inserted into the cell or organism. For bacteria or eukaryotic cells in culture, this is usually in the form of a plasmid. Commonly used reporter genes may include fluorescent proteins, luciferase, beta-galactosidase, and selectable markers, such as chloramphenicol, and ccdB.

The term “cDNA” refers to DNA synthesized from a mature mRNA template. cDNA is most often synthesized from mature mRNA using the enzyme reverse transcriptase. The enzyme operates on a single strand of mRNA, generating its complementary DNA based on the pairing of RNA base pairs (A, U, G, C) to their DNA complements (T, A, C, G). There are several methods known for generating cDNA, for example, to obtain eukaryotic cDNA whose introns have been spliced: a) an eukaryotic cell transcribes the DNA (from genes) into RNA (pre-mRNA); b) the same cell processes the pre-mRNA strands by splicing out introns, and adding a poly-A tail and 5′ methyl-guanine cap; c) this mixture of mature mRNA strands are extracted from the cell; d) a poly-T oligonucleotide primer is hybridized onto the poly-A tail of the mature mRNA template (reverse transcriptase requires this double-stranded segment as a primer to start its operation.); e) reverse transcriptase is added, along with deoxynucleotide triphosphates (A, T, G, C); and f) the reverse transcriptase scans the mature mRNA and synthesizes a sequence of DNA that complements the mRNA template. This strand of DNA is complementary DNA. (see also Current Protocols in Molecular Biology, John Wiley & Sons).

The term “cloning host cell” refers to a host cell that contains a cloning vector.

The term “cloning vector” refers to a DNA molecule such as a plasmid, cosmid, or bacterial phage, or virus, such as, for example retroviruses, adeno-associated adenoviruses, lentivirus, baculoviruses and adenoviruses, that has the capability of replicating autonomously in a host cell. Cloning vectors typically contain one or a small number of restriction endonuclease recognition sites at which foreign DNA sequences can be inserted in a determinable fashion without loss of essential biological function of the vector, as well as a selectable marker gene that is suitable for use in the identification and selection of cells transformed with the cloning vector. Selectable marker genes may include genes that provide tetracycline resistance. ampicillin resistance, or other observable features, such as with the ccdB gene.

The term “detectable marker” encompasses both the selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. When the nucleic acid is prepared or altered synthetically, advantage can be taken of known codon preferences of the intended host where the nucleic acid is to be expressed.

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid molecules and enzymes. To facilitate detection, a detection reagent may comprise a marker.

The term “DNA recombination sequence” refers to a nucleic acid sequence that provides for efficient transfer of DNA fragments across multiple systems and into multiple vectors. Any DNA fragment flanked by a recombination site can be transferred into any vector that has a corresponding site. Orientation and reading frame are maintained with efficiency (typically 99%), effectively eliminating the need for secondary sequencing or subcloning after the initial entry clone is made. The transfer of DNA fragments makes use of lambda phage-based site-specific recombination instead of restriction endonuclease and ligase to insert a gene of interest into an expression vector. The DNA recombination sequences, for example, attL, attR, attB, and attP, and enzyme mixtures, for example, LR and BP Clonase™, may be used to mediate the lambda recombination reactions. Transferring a gene into a destination vector is accomplished in two steps: 1) clone the gene of interest into an entry vector and 2) mix the entry clone containing the gene of interest in vitro with the appropriate expression vector (destination vector) and enzyme mix. Site-specific recombination between the att sites (attR×attL attB×attP) generates an expression clone and a by-product. The expression clone contains the gene of interest recombined into the destination vector backbone. Following transformation and selection in E. coli, the expression clone is ready to be used for expression in the appropriate host. This lambda-based system is also known as the Gateway® cloning system (Invitrogen Inc., Carlsbad, Calif.).

The term “electroporation” refers to a significant increase in the electrical conductivity and permeability of the cell plasma membrane caused by an externally applied electrical field. It is used as a way of introducing some substance into a cell, such as loading it with a piece of coding DNA, a molecular probe, or a drug. Pores are formed when the voltage across a plasma membrane exceeds its dielectric strength. If the strength of the applied electrical field and/or duration of exposure to it are properly chosen, the pores formed by the electrical pulse reseal after a short period of time, during which extracellular compounds have a chance to enter into the cell. However, excessive exposure of live cells to electrical fields can result in cell death. Electroporation is done with electroporators, instruments which create the electric current and send it through the cell solution, typically bacteria. The solution is pipetted into a glass or plastic cuvette which has two Al electrodes on its sides. For example, for bacterial electroporation, a suspension of around 50 μl is usually used. Prior to electroporation, it is mixed with the plasmid to be transformed. The mixture is pipetted into the cuvette, the voltage is set on the electroporator (2,400 volts is often used) and the cuvette is inserted into the electroporator and an electric current is applied. Immediately after electroporation 1 ml of liquid medium is added to the bacteria (in the cuvette or in a microcentrifuge tube), and the tube is incubated at the bacteria's optimal temperature for an hour or more and then it is spread on an agar plate (see Ausubel, Current Protocols in Molecular Biology, Wiley).

The term “equimolar” refers to having an equal concentration of moles in one liter of solution.

The term “expression system” refers to a genetic sequence which includes a protein encoding region which is operably linked to all of the genetic signals necessary to achieve expression of the protein encoding region. Traditionally, the expression system will include a regulatory element such as a promoter or enhancer, to increase transcription and/or translation of the protein encoding region, or to provide control over expression. The regulatory element may be located upstream or downstream of the protein encoding region, or may be located at an intron (non coding portion) interrupting the protein encoding region. Alternatively it is also possible for the sequence of the protein encoding region itself to comprise regulatory ability.

The term “expression vector” refers a DNA molecule comprising a gene that is expressed in a host cell. Typically, gene expression is placed under the control of certain regulatory elements including promoters, tissue specific regulatory elements, and enhancers. Such a gene is said to be “operably linked to” the regulatory elements.

The term “functional splice acceptor” refers to any individual functional splice acceptor or functional splice acceptor consensus sequence that permits the construct of the disclosure to be processed such that it is included in any mature, biologically active mRNA, provided that it is integrated in an active chromosomal locus and transcribed as a contiguous part of the pre-messenger RNA of the chromosomal locus.

The term “homing endonucleases” refers to double stranded DNases that have large, asymmetric recognition sites (12-40 base pairs) and coding sequences that are usually embedded in either introns or inteins. Introns are spliced out of precursor RNAs, while inteins are spliced out of precursor proteins. Homing endonucleases are named using conventions similar to those of restriction endonucleases with intron-encoded endonucleases containing the prefix, “I-” and intein endonucleases containing the prefix, “PI-”. Homing endonuclease recognition sites are extremely rare. For example, an 18 base pair recognition sequence will occur only once in every 7×10¹⁰ base pairs of random sequence. This is equivalent to only one site in 20 mammalian-sized genomes. However, unlike standard restriction endonucleases, homing endonucleases tolerate some sequence degeneracy within their recognition sequence. As a result, their observed sequence specificity is typically in the range of 10-12 base pairs. Homing endonucleases do not have stringently-defined recognition sequences in the way that restriction enzymes do. That is, single base changes do not abolish cleavage but reduce its efficiency to variable extents. The precise boundary of required bases is generally not known.

The term “host cell” encompasses any cell which contains a vector and preferably supports the replication and/or expression of the vector. Host cells may be prokaryotic cells such as Escherichia coli, or eukaryotic cells such as yeast, insect, amphibian, or mammalian cells. The term as used herein means any cell which may be in culture or in vivo as part of a unicellular organism, part of a multicellular organism, or a fused or engineered cell culture.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed.

The term “internal ribosome entry site” (IRES) refers to an element which permits attachment of a downstream coding region or open reading frame with a cytoplasmic polysomal ribosome for purposes of initiating translation thereof in the absence of any internal promoters. An IRES is included to initiate translation of selectable marker protein coding sequences. Examples of suitable IRESes that can be used include the mammalian IRES of the immunoglobulin heavy-chain-binding protein (BiP). Other suitable IRESes are those from the picornaviruses. For example, such IRESes include those from encephalomyocarditis virus (preferably nucleotide numbers 163-746), poliovirus (preferably nucleotide numbers 28-640) and foot and mouth disease virus (preferably nucleotide numbers 369-804). Thus, the IRES are located in the long 5′ untranslated regions of the picornaviruses which can be removed from their viral setting in length to unrelated genes to produce polycistronic mRNAs.

The term “isolated” refers to material, such as a nucleic acid or a protein, which is: (1) substantially or essentially free from components that normally accompany or interact with it as found in its naturally occurring environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein are also referred to as “heterologous” nucleic acids. The isolated material optionally comprises material not found with the material in its natural environment

The term “inserted” or “introduced” in the context of inserting a nucleic acid into a cell, refers to “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “label” or “labeled” refers to incorporation of a detectable marker or molecule, e.g., by incorporation of a radiolabeled nucleoside triphosphates or radioisotopes to a nucleic acid that can be detected or measured. Various methods of labeling nucleic acids are known in the art (see Short Protocols in Molecular Biology, 5^{th} Ed., John Wiley & Sons, 2002) and may be used. Examples of labels for nucleic acids include, but are not limited to, the following: radioisotopes (e.g., ³²P-labeled NTPs and dNTPs; ³⁵S-labeled NTPs and dNTPs; ³H′ ¹⁴C; ¹²⁵I), fluorophores and fluorescent labels (e.g., FITC; rhodamine; lanthanide phosphors; cyanine (Cy3, Cy5); fluorescein; coumarin, SYBR Green); and digoxygenin-11-dUTP.

The term “MA segment”, also referred to as a “MA sequence,” refers to a nucleotide sequence located downstream from the TAG and upstream of the transcription termination signal in the TAG plasmids and their derivatives. All mRNAs synthesized from the various promoters studied in a single experiment will contain the same MA sequence, to which a complementary primer can anneal and initiate the synthesis of the first strand cDNA in order to make hybridization probes. The MA sequence is usually 20 to 30 nucleotides in length, but may be longer provided the MA sequence does not contain any secondary structure, such as hairpin loops, which would prevent an efficient cDNA synthesis. The MA sequence is composed of approximately 50% GC, such that the melting temperature ranges from about 70° C. to about 75° C. MA sequences are unique among all published nucleotide databases, so that only the TAG-transcripts will serve as template for cDNA synthesis. MA sequences do not contain any of the restriction sites that are used elsewhere in the TAG plasmids for cloning purposes. It cannot function as (or does not contain) a transcriptional promoter or transcription termination signal.

The term “mixing” refers to combining, joining, uniting, associating, fusing, or ligating at least two distinct nucleotide sequences such that they become one fragment.

The term “multiple cloning site,” also referred to as an “MCS” or a “polylinker” refers to a short segment of DNA which contains many (usually 20+) sites recognized by restriction enzymes or other endonucleases such as homing endonucleases.

The term “nucleic acid” refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” refers to a chemical compound that consists of a heterocyclic base, a sugar, and one or more phosphate groups. In the most common nucleotides the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors: CoA, FAD, DMN, NAD, and NADP. The purines include adenine (A), and guanine (G); the pyrimidines include cytosine (C), thymine (T), and uracil (U).

The terms “oligoclonal” and/or “polyclonal” applied to cell populations indicates a population of cells where some cells within that population are not genetically identical to the rest of the cells of that population. Conversely, the term “monoclonal” or “monoclonal cell population” indicates that all cells within that population are genetically identical. Differences in the “genetic identity” of a population of cells in the context of this disclosure arise by random retroviral integration into different genomic insertion sites.

The term “operably linked” refers to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The term “optical density” refers to the absorbance of an optical element for a given wavelength per unit distance. Typically, bacterial cultures are measured at a wavelength of 600 nm.

The term “polymerase chain reaction” or “PCR” refers to a procedure described in U.S. Pat. No. 4,683,195, the disclosure of which is incorporated herein by reference.

The term “polynucleotide” refers to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma.-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

The term “primer” refers to a nucleic acid which, when hybridized to a strand of DNA, is capable of initiating the synthesis of an extension product in the presence of a suitable polymerization agent. The primer preferably is sufficiently long to hybridize uniquely to a specific region of the DNA strand. A primer may also be used on RNA, for example, to synthesize the first strand of cDNA.

The term “promoter” refers to a region of DNA upstream, downstream, or distal, from the start of transcription and involved in recognition and binding of RNA polymerase and other proteins to initiate transcription. For example, T7, T3 and Sp6 are RNA polymerase promoter sequences. In RNA synthesis, promoters are a means to demarcate which genes should be used for messenger RNA creation and by extension, control which proteins the cell manufactures. Promoters represent critical elements that can work in concert with other regulatory regions (enhancers, silencers, boundary elements/insulators) to direct the level of transcription of a given gene.

The term “promoter sequence candidate” refers to a nucleotide sequence that contains a putative promoter sequence. A promoter sequence candidate may be provided by a computer-predicted model, DNA fragments from a collection of nucleotide sequences, such as a genomic library, deletion or site-directed mutants of a specific promoter, tissue-specific promoters, artificial promoters, etc.

The term “promoterless” refers to a protein coding sequence contained in a vector, retrovirus, adenovirus, adeno-associated virus or retroviral provirus that is not directly or significantly under the control of a promoter within the vector, whether it be in RNA or DNA form. The vector, plasmid, viral or otherwise, may contain a promoter, but that promoter cannot be positioned or configured such that it directly or significantly regulates the expression of the promoterless protein coding sequence.

The term “protein coding sequence” refers a nucleotide sequence encoding a polypeptide gene which can be used to distinguish cells expressing the polypeptide gene from those not expressing the polypeptide gene. Protein coding sequences include those commonly referred to as selectable markers. Examples of protein coding sequences include those coding a cell surface antigen and those encoding enzymes. A representative list of protein coding sequences include thymidine kinase, beta.-galactosidase, tryptophan synthetase, neomycin phosphotransferase, histidinol dehydrogenase, luciferase, chloramphenicol acetyltransferase, dihydrofolate reductase (DHFR); hypoxanthine guanine phosphoribosyl transferase (HGPRT), CD4, CD8 and hygromycin phosphotransferase (HYGRO).

The term “recombinant” refers to a cell or vector that has been modified by the introduction of a heterologous nucleic acid or the cell that is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation transduction/transposition) such as those occurring without deliberate human intervention.

The term “recombinant expression cassette” refers to a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The term “recombinant host” refers to any prokaryotic or eukaryotic cell that contains either a cloning vector or an expression vector. This term also includes those prokaryotic or eukaryotic cells that have been genetically engineered to contain the cloned genes, or gene of interest, in the chromosome or genome of the host cell.

The term “regulatory sequence” (also called regulatory region or regulatory element) refers to a promoter, enhancer or other segment of DNA where regulatory proteins such as transcription factors bind preferentially. They control gene expression and thus protein expression.

The term “reporter cell line” refers to prokaryotic or eukaryotic cells that contain a reporter or assay marker.

The term “restriction digestion” refers to a procedure used to prepare DNA for analysis or other processing. Also known as DNA fragmentation, it uses a restriction enzyme to selectively cleave strands of DNA into shorter segments.

The term “restriction enzyme” (or restriction endonuclease) refers to an enzyme that cuts double-stranded DNA. The enzyme makes two incisions, one through each of the phosphate backbones of the double helix without damaging the bases. Restriction enzymes are classified biochemically into four types, designated Type 1, Type II, Type III, and Type IV. In Type I and Type III systems, both the methylase and restriction activities are carried out by a single large enzyme complex. Although these enzymes recognize specific DNA sequences, the sites of actual cleavage are at variable distances from these recognition sites, and can be hundreds of bases away. Both require ATP for their proper function. In Type II systems, the restriction enzyme is independent of its methylase, and cleavage occurs at very specific sites that are within or close to the recognition sequence. Type II enzymes are further classified according to their recognition site. Most Type II enzymes cut palindromic DNA sequences, while Type IIa enzymes recognize non-palindromic sequences and cleavage outside of the recognition site. Type IIb enzymes cut sequences twice at both sites outside of the recognition sequence. In Type IV systems, the restriction enzymes target only methylated DNA.

The term “restriction sites” or “restriction recognition sites” refer to particular sequences of nucleotides that are recognized by restriction enzymes as sites to cut the DNA molecule. The sites are generally, but not necessarily, palindromic, (because restriction enzymes usually bind as homodimers) and a particular enzyme may cut between two nucleotides within its recognition site, or somewhere nearby.

The term “reverse transcription” or “reverse transcription polymerase chain reaction” (RT-PCR) refers to amplifying a defined piece of a ribonucleic acid (RNA) molecule. The RNA strand is first reverse transcribed into its DNA complement or complementary DNA, followed by amplification of the resulting DNA using polymerase chain reaction.

The term “selectable marker” refers to a gene introduced into a cell, especially a bacterium or to cells in culture that confers a trait suitable for artificial selection. They are a type of reporter gene used in laboratory microbiology, molecular biology, and genetic engineering to indicate the success of a transfection or other procedure meant to introduce foreign DNA into a cell. For example, analysis of gene function frequently requires the formation of cells that contain the studied gene in a stably integrated form. In some situations, few cells may stably integrate DNA thus a dominant selectable marker is used to permit isolation of stable transfectants. Selectable markers may include: antibiotics (ampicillin) and ‘suicide’ genes (for example ccdB). Positive selective markers may utilize: adenosine deaminase (thymidine, hypoxanthine, 9-β-D-xylofuranosyl adenine, 2′-deoxycoformycin), aminoglycoside phosphotransferase (neomycin, G418, gentamycin, kanamycin), Bleomycin (bleomycin, phleomycin, zeocin), cytosine deaminase (N-(phosphonacetyl)-L-aspartate, inosine, cytosine); dehydrofolate reductase (methotrexate, aminopterin); histidinol dehydrogenase (histindol); hygromycin-B-phosphotransferase (hygromycin-B); puromycin-N-acetyl transferase (puromycin); thymidine kinase (hypoxanthine, aminopterin, thymidine, glycine); and xanthine-guanine phosphorriobsyltransferase (xanthine, hypoxanthine, thymidine, aminopterin, mycophenolic acid, L-glutamine). Negative selectable markers may utilize: cytosine deaminase (5-fluorocytosine); diptheria toxin; ccdB, and HSV-TK.

The term “selectively hybridizes” refers to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have at least about 80% sequence identity, preferably at least 90% sequence identity, and most preferably 100% sequence identity (i.e., complementary) with each other.

The term “sense” refers to the general concept used to compare the polarity of nucleic acid molecules to other nucleic acid molecules. Generally, a DNA sequence is called “sense” if its sequence is the same as that of a messenger RNA copy that is translated into protein. The sequence on the opposite strand is complementary to the sense sequence and is therefore called the “antisense” sequence.

The term “TAG” refers to a DNA sequence composed of random nucleotides, in which each position has an equal probability of having any of the four deoxynucleotides (A, C, T, and G). Other bases, such as inosine, uracil, 5-methylcytosine, 8-azaguanine, 2,6-diaminopurine, 5 bromouracil, and other derivatives may be incorporated in their nucleotide form into the sequences. The length of the TAG sequence is short, for example between about 16 by to about 200 bp, between about 20 to about 150 bp, between about 30 to about 120 bp, between about 40 to about 100 bp, between about 50 to about 75 bp, or about 65, 60 or 21 bp. The sequences are preferably different or distinct enough to avoid annealing to each other at times when the oligonucleotide is present as a single strand, and lack any significant homology to known sequences. In addition, the sequence should not be self-complementary, so as to avoid the formation of primer-dimers during amplification. Within a plurality of TAG sequences, each TAG sequence will have approximately equivalent amounts of the nucleotides A, T, G, and C such that each TAG sequence has approximately the same melting temperature as the other TAGs. A same melting temperature will allow for the unbiased quantification of various mRNAs containing each a different TAG sequence by hybridization under the same temperature and ionic strength conditions. Within a plurality of TAG sequences, the nucleotide sequence of each individual TAG sequence is unique to the individual TAG of the plurality.

The term “transcription termination signal” refers to a section of genetic sequence that marks the end of gene or operon on genomic DNA for transcription. In prokaryotes, two classes of transcription termination signals are known: 1) intrinsic transcription termination signals where a hairpin structure forms within the nascent transcript that disrupts the mRNA-DNA-RNA polymerase ternary complex; and 2) Rho-dependent transcription termination signal that require Rho factor, an RNA helicase protein complex to disrupt the nascent mRNA-DNA-RNA polymerase ternary complex. In eukaryotes, transcription termination signals are recognized by protein factors that co-transcriptionally cleave the nascent RNA at a polyadenlyation signal (i.e, “poly-A signal” or “poly-A tail”) halting further elongation of the transcript by RNA polymerase. The subsequent addition of the poly-A tail at this site stabilizes the mRNA and allows it to be exported outside the nucleus. Termination sequences are distinct from termination codons that occur in the mRNA and are the stopping signal for translation, which may also be called nonsense codons.

The term “translational stop sequence” refers to a sequence which codes for the translational stop codons. In some embodiments, the translational stop sequence may be in one, two, or three reading frames.

The term “transfection” refers to the introduction of foreign DNA into eukaryotic or prokaryotic cells. Transfection typically involves opening transient holes in cells to allow the entry of extracellular molecules, typically supercoiled plasmid DNA, but also siRNA, among others. There are various methods of transfecting cells. One method is by calcium phosphate. HEPES-buffered saline solution containing phosphate ions is combined with a calcium chloride solution containing the DNA to be transfected. When the two are combined, a fine precipitate of calcium phosphate will form, binding the DNA to be transfected on its surface. The suspension of the precipitate is then added to the cells to be transfected. The cells take up precipitate and the DNA. Alternatively, MgCl₂ or RbCl can be used. Other methods of transfection include electroporation, heat shock, proprietary transfection agents, dendrimers, and the use of liposomes. Liposomes are small, membrane-bounded bodies that fuse to the cell membrane releasing DNA into the cell. For eukaryotic cells, lipid-cation based transfection is typically used. Other methods of transfection include use of the gene gun and viruses. For stable transfection another gene is co-transfected, which gives the cell some selection advantage, such as resistance towards a certain toxin. If the toxin, towards which the co-transfected gene offers resistance, is then added to the cell culture, only those cells with the foreign genes inserted into their genome will be able to proliferate, while other cells will die. After applying this selection pressure for some time, only the cells with a stable transfection remain and can be cultivated further. A common agent for stable transfection is Geneticin, also known as G418, which is a toxin that can be neutralized by the product of the neomycin resistant gene (see Bacchetti and Graham. Transfer of the gene for thymidine kinase to thymidine kinase-deficient human cells by purified herpes simplex viral DNA. 1977. Proc. Natl. Acad. Sci. USA 74(4):1590-94). Conventional transient transfection assays may incorporate internal controls, such as pRL-SV40 (Promega, Inc.) and may be used in combination with any experimental reporter vector to co-transfect mammalian cells.

The term “transformation” refers to the genetic alteration of a cell resulting from the introduction, uptake, and expression of foreign genetic material (DNA or RNA). In bacteria, transformation refers to a genetic change brought about by taking up and expressing DNA, and “competence” refers to a state of being able to take up DNA. Competent cells may be generated by a laboratory procedure in which cells are passively made permeable to DNA, using conditions that do not normally occur in nature, thus cells that have been manipulated to accept foreign DNA are called “competent cells”. These procedures are comparatively easy and simple, and can be used to genetically engineer bacteria. These procedures may include chilling cells in the presence of divalent cations, such as CaCl₂, which prepares the cell walls to become permeable to plasmid DNA. Cells are incubated with the DNA and then briefly heat shocked (e.g., 42° C. for 30-120 seconds), which causes the DNA to enter the cell. This method works well for circular plasmid DNAs. Electroporation is another way to allow DNA to enter cells and involves briefly shocking cells with an electric field of 100-200 V. Plasmid DNA enters cells via the holes created in the cell membrane by the electric shock; natural membrane-repair mechanisms close these holes afterwards. Yeasts may be transformed, for example, by High Efficiency Transformation (see Gietz, R. D., and R. A. Woods. 2002 Transformation of Yeast by the Liac/SS Carrier DNA/PEG Method. Methods in Enzymology 350:87-96); the Two-hybrid System Protocol (see Gietz, R. D., B. Triggs-Raine, A. Robbins, K. C. Graham, and R. A. Woods. 1997 Identification of proteins that interact with a protein of interest: Applications of the yeast two-hybrid system. Mol Cell Biochem 172:67-79); and the Rapid Transformation Protocol (see Gietz, R. D., and R. A. Woods. 2002 Transformation of Yeast by the Liac/SS Carrier DNA/PEG Method. Methods in Enzymology 350:87-96).

The term “vector” refers to a nucleic acid used in transfection of a host cell and into which can be inserted a polynucleotide. Vectors are frequently replicons. Expression vectors permit transcription of a nucleic acid inserted therein. Some common vectors include plasmids, cosmids, viruses, phages, recombinant expression cassettes, and transposons. The term “vector” may also refer to an element which aids in the transfer of a gene from one location to another. Vectors may include expression vectors and cloning vectors.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”. The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

All TAGs should lack homology to other TAGs used within the same assay. Dependent upon the method by which the probe is made, homology of the TAG with known nucleic acid sequences may be acceptable. For example, if the probe is made by labeling mRNA directly, for example with polyA polymerase (see, for example, Aviv and Leder, Proc. Natl. Acad. Sci. USA. 1972 June; 69(6):1408-12), the TAG-containing mRNAs, the endogenous mRNAs and possibly the tRNA, and rRNA may be labeled as well. Hybridization by these latter RNAs may interfere with detection by the probe. The TAGs should not have homology with any known sequence that is transcribed into RNA, including mRNA, tRNA, rRNA, etc. If the probe is made by labeling the first-strand cDNA, there are two possibilities: 1) if oligo(dT) is used as a primer, all first strand cDNA synthesized from mRNAs will be labeled, including the TAG-containing mRNAs and the endogenous mRNAs. These latter cDNAs may interfere with detection by the probe, thus the TAGs should not have homology with any known sequence that is transcribed into RNA; and 2) if oligo(dT)+anchor is used as a primer “B” (where the anchor would be a short stretch of nucleotides corresponding to the 3′ end of the mRNA, immediately preceding the polyA) only cDNAs synthesized from mRNAs terminated by the same or similar transcription termination signal as the one used for the TAG constructs will be labeled. Thus if a particular kind of endogenous mRNA is recognized by the oligo(dT)-anchor primer, that specific mRNA would interfere with detection by the probe, therefore the TAG should not share homology with that specific mRNA. If the probe is made by PCR, in addition to the homology considerations discussed above with regard to the synthesis of the first strand cDNA, there are two additional considerations. First, linear amplification of the first strand cDNA is made using a primer (A) corresponding to a region common to all the TAG-mRNAs that is located 5′ to the TAG. This situation may arise when the vector (plasmid or viral DNA), from which the probe may be made from, is removed and the primer B used for the first strand cDNA synthesis is removed as well. Accordingly, if the first strand cDNA was synthesized using oligo(dT) as the primer, then the TAGs may not have homology with any known sequence that is transcribed into mRNA, and that shares sequence identity with primer A, and if the first strand cDNA was synthesized using oligo(dT)-anchor as the primer, then the TAGs may not have homology with any known sequence that is transcribed into mRNA that shares sequence identity with both the 3′ end as the TAG-mRNA and primer A. Second, exponential amplification of the first strand cDNA using primer (A) and the oligo(dT)-based primer occurs. In this situation, the antisense strand may be used as a probe and the printing of the assay membrane with the sense-strand oligonucleotides so that the vector does not have to be removed, as discussed above. Thus, at times, one can use TAGs with sequences that are found elsewhere in databases. A specific TAG should not share sequence homology with any other TAG used simultaneously in the same assay and with any DNA or RNA molecule that will be labeled during the synthesis of the probe, regardless of the method used to synthesize the probe.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination. As used herein, “sequence identity” or “identity” in the context of two nucleic acid or polypeptide sequences refers to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%. Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid. The terms “substantial Identity” in the context of a peptide indicates that a peptide comprises a sequence with at least 70% sequence identity to a reference sequence, preferably 80%, ore preferably 85%, most preferably at least 90% or 95% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

Methods of extraction of RNA are well-known in the art and are described, for example, in J. Sambrook et al., “Molecular Cloning: A Laboratory Manual” (Cold Spring Harbor Laboratory Press, Cold Spring Harbor, N.Y., 1989), vol. 1, ch. 7, “Extraction, Purification, and Analysis of Messenger RNA from Eukaryotic Cells,” incorporated herein by this reference. Other isolation and extraction methods are also well-known, for example in F. Ausubel et al., “Current Protocols in Molecular Biology, John Wiley & Sons). Typically, isolation is performed in the presence of chaotropic agents such as guanidinium chloride or guanidinium thiocyanate, although other detergents and extraction agents can alternatively be used. Typically, the mRNA is isolated from the total extracted RNA by chromatography over oligo(dT)-cellulose or other chromatographic media that have the capacity to bind the polyadenylated 3′-portion of mRNA molecules. Alternatively, but less preferably, total RNA can be used. However, it is generally preferred to isolate poly(A)+RNA.

The method employs several basic steps to achieve its objective. First, a library of DNA TAGs is designed. The DNA TAG sequences are composed of random nucleotides. Each DNA TAG sequence, in one embodiment of approximately 60 by in length, is unique among a plurality of TAG sequences, i.e. a specific TAG does not share sequence homology with any other TAG used simultaneously in the same assay and with any DNA or RNA molecule that will be labeled during the synthesis of the probe, regardless of the method used to synthesize the probe. The TAG sequences have similar physical properties so that a plurality of the TAG sequences can be used for hybridization under similar conditions. Second, pTAG-basic plasmids are constructed. Third, the TAG sequences are inserted into the pTAG-basic plasmids. Fourth, promoter array membranes are prepared. Fifth, promoter sequence candidates are inserted into the pTAG plasmids. Sixth, the pTAG plasmids with the promoter sequence candidate inserts are transfected into host cells, and the RNA extracted. The RNA or the resultant cDNA derived from the extracted RNA is then labeled, hybridized to the promoter array membrane, and analysis performed. Thus, the present disclosure discloses an array-based method for promoter detection and analysis. The method provides for transcriptional products that are tagged as they are synthesized, in such a way that one specific transcript is labeled with only one type of TAG, and one TAG labels only one type of transcript. All promoter sequence candidates are analyzed simultaneously in one reaction vial. The transcriptional output is analyzed on conventional arrays.

### BRIEF DESCRIPTION OF THE DRAWINGS

FIG. 1. Flow diagram of a first array-based promoter detection and analysis method disclosed herein. 1=PROMOTER CANDIDATES; 2=TAG-PLASMIDS; 3=POOL ALL E. COLI CLONES AT THE SAME CELL DENSITY, GROW AND PURIFY PLASMID; 4=TRANSFECT PLASMID INTO CELLS; 5=EXTRACT TOTAL RNA, LABEL and AMPLIFY cDNA PROBES; 6=DETECT CLONES WITH PROMOTER ACTIVITY.

FIG. 2. BrightStar®-Plus membranes spotted manually (left) or using a robot (right) with a collection of reverse-strand TAG oligonucleotides.

FIGS. 3A and 3B. Comparative analysis of the activity of 42 promoters in a single population of HEK 293 cells. The 42 promoter-TAG plasmids and 8 promoter-less TAG-reporter plasmids were mixed in equimolar amounts and transfected into the same cell population. Total RNA was extracted 14 hours after transfection. RNA was labeled using the linear amplification method, and biotin-labeled probes were hybridized on the TAG-spotted membranes (FIG. 3A). Hybridization was revealed by chemiluminescence, and quantified by densitometry (FIG. 3B). The macro array membrane was made by spotting manually each oligonucleotide as a diagonal doublet.

FIGS. 4A and 4B. Comparison of the transcriptional activities of 92 promoters in a single cell population. The 92 promoter-TAG plasmids and 8 promoter-less TAG-reporter plasmids were mixed in equimolar amounts and transfected into the same cell population. Total RNA was extracted 14 hours after transfection. RNA was labeled using the linear amplification method, and biotin-labeled probes were hybridized on the TAG-spotted membranes (FIG. 4A). Hybridization was revealed by chemiluminescence, and quantified by densitometry (plain bars) (FIG. 4B). The relative luciferase activities obtained with each plasmid construct were obtained from previously published work and are shown at the bottom (empty bars) (FIG. 4B). The numbers at the bottom of the figure refer to the list of promoters described in Table 1. The luciferase data obtained with the various OM promoters (# 59-73), defensin promoters (# 74-85), and other promoters studied by Coleman (Coleman, S., et al. Experimental analysis of the annotation of promoters in the public database. Hum. Mol. Genet., 2002. 11(16): 1817-1821) were generated in different experimental conditions and should not be compared between each other. The macroarray membrane was made by spotting each oligonucleotide as a quadruplet, using a Biorobotics MicroGrid array spotting robot (Genomic Solutions, Ann Arbor, Mich.) at the microarray facility of the University of Idaho Environmental Biotechnology Institute (Moscow, ID).

FIGS. 5A and 5B. Validation of the Promoter Detective method with a set of 35 promoter-TAG plasmids. The autoradiogram (FIG. 5A) was obtained by hybridizing radioactive TAG-cDNA probes to a membrane spotted with the complementary TAG strands. The identity of the spots is indicated by numbers on the left side of the autoradiogram, and on the bottom of the bar chart (FIG. 5B). The bar chart summarizes the intensities of the various spots, relative to the signal obtained with the CMV promoter (=100).

FIG. 6A-G. Flow diagram for the construction of the pTAG reporter plasmid. FIG. 6A shows pGL4.7=SfiI, BglI, Acc651, KpnI, SacI, NheI, XhoI, EcoRV, BglI, SfiI; 8=HindIII; 9=NcoI; 10=ApaI; 11=XbaI; 23=Amp; 24=Luc2; 25=polyA; 11=XbaI. pGL4 is cut with SfiI and insert linker to yield pGL4-12.

FIG. 6B shows pGL4-12.13=EcoRI, KpnI, SacI, NheI, XhoI, BglII, SfiI, BgII, SmaI, BgII, SfiI; 8=HindIII; 9=NcoI; 10=ApaI; 23=AMP; 24=Luc2; 25=polyA; 11=XbaI. pGL4-12 is cut with XhoI/BglII and insert linker to yield pGL4-1256.

FIG. 6C shows pGL4-1256. 15=EcoRI, KpnI, SacI, NheI, XhoI, BglII, ApaI, NruI, KpnI, XhoI, SacI, BglII, NheI, EcoRV, MluI, SfiI, BglI, SmaI, BglI SfiI; 8=HindIII; 9=NcoI; 10=ApaI; 23=Amp; 24=Luc2; 25=polyA. pGL4-1256 is cut with NcoI/XbaI and insert MA4 to yield 1256MA4.

FIG. 6D shows 1256MA4. 15=EcoRI, KpnI, SacI, NheI, XhoI, BglII, ApaI, NruI, KpnI, XhoI, SacI, BglII, NheI, EcoRV, MluI, SfiI, BglI, SmaI, BglI, SfiI; 8=HindIII; 9=NcoI; 10=ApaI; 25=polyA; 23 Amp; 26=MA4. 1256MA4 is cut with EcoRV/MluI, insert T7 to yield 1256MA4T7.

FIG. 6E shows 1256MA4T7. 18=EcoRI, KpnI, SacI, NheI, XhoI, BglII, ApaI, NruI, KpnI, XhoI, SacI, BglII, NheI, EcoRV; 19=MluI, SfiI, BglI, SmaI, BglI, SfiI. HindIII; 9=NcoI; 10=ApaI; 25=polyA; 23=Amp; 27=T7. 1256MA4T7 is cut with ApaI and insert attP to yield 1256MA4T7att.

FIG. 6F shows 1256MA4T7att. 21=EcoRI, KpnI, SacI, NheI, XhoI, BglII, ApaI; 28=attP2; 48=EcoRI; 29=ccdB; 48=EcoRI; 9=NcoI; 49=SmaI; 51=attP1; 22=RV, NheI, BglII, SacI, XhoI, KpnI, NruI, ApaI; 27=T7; 59=HindIII, SfiI, BlgI, SmaI, BlgI, SfiI, MluI; 53=NcoI; 55=MluI; 56=TAG; 54=HindIII; 26=MA4; 52=XbaI; 25=polyA; 23=Amp; 1256MA4T7att is cut with SfiI and insert TAG to yield pTAG reporter.

FIG. 6G shows pTAG reporter. 21=EcoRI, KpnI, SacI, NheI, XhoI, BglII, ApaI; 28=attP2; 48=EcoRI; 29=ccdB; 9=NcoI; 49=SmaI; 51=attP1; 22=RV, NheI, BglII, SacI, XhoI, KpnI, NruI, ApaI; 27=T7; 55=MluI; 56=TAG; 54=HindIII; 53=NcoI; 26=MA4; 52=XbaI; 25=polyA; 23=Amp; 59=HindIII, SfiI, BlgI, SmaI, BlgISfiI, MluI.

FIG. 7. Plasmid map of the pTAG basic vector. 30=PROMOTER; 31=attP2; 32=MCS2 60=SfiI; 61=TAG (5′UTR); 62=ATG; 63=Luciferase cDNA; 64=α-Globin 3′ UTR; 65=SV40 late poly(A) signal; 66=ORI; 67=Amp; 68=Kan; 69=Synthetic poly (A) signal; 70=MCS1; 71=attP1; 72=ccdB (promoter); 73=attP2; 74=MCS2.

FIG. 8. Flow diagram of a second array-based promoter detection and analysis method disclosed herein. The letter P represents a TaqMan probe; A, B and C represent unique TAG sequences; X, Y and Z represent candidate sequences under investigation, and the grey box represents an RNA adaptor sequence.

TABLE 1. List of 100 promoter sequences used within the examples. Each promoter is described with its symbol, length, and Refseq or GenBank accession number. The TAG identification number to which it is associated is also indicated.

### DETAILED DESCRIPTION

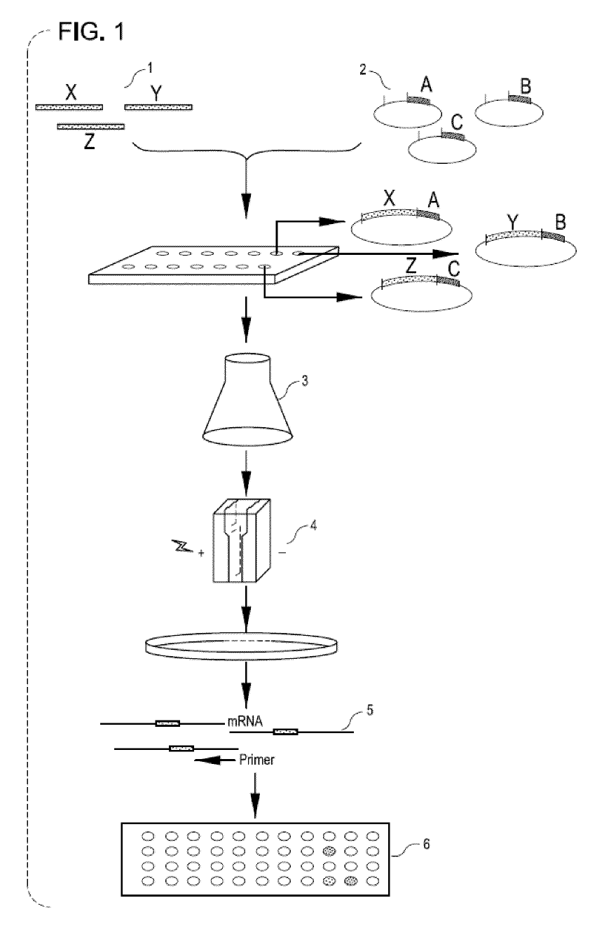

The present disclosure provides methods for the detection and analysis of DNA promoter sequences. FIG. 1 provides a general flow chart of one of the disclosed methods. In this embodiment, a vector library is constructed that contains potential DNA regulatory or promoter sequence candidates that may be present, for example, in a collection of nucleotide sequences, such as a genomic library, in computer-predicted promoter regions, or in deletion mutants of promoters under investigation, etc. Each clone generated potentially drives the transcription of a unique reporter gene composed of a well-defined, approximately 60-bp long DNA TAG sequence composed of random nucleotides. The transcriptional properties of the various constructs are analyzed by pooling equimolar amounts of vectors and transfecting them into a cell line of interest. RNA is extracted, cDNA synthesized and labeled, directly or indirectly, and quantified by hybridization to the DNA TAGs arrayed on a membrane, glass, or bead support. Suitable bead compositions include those used in peptide, nucleic acid and organic moiety synthesis, including but not limited to, plastics, ceramics, glass, polystyrene, methylstyrene, acrylic polymers, paramagnetic materials, thoria sol, carbon graphite, titanium dioxide, latex or cross-linked dextrans such as sepharose, cellulose, nylon, cross-linked micelles and Teflon™ (see Microsphere Detection Guide, Bangs Laboratories, Fishers Ind.).

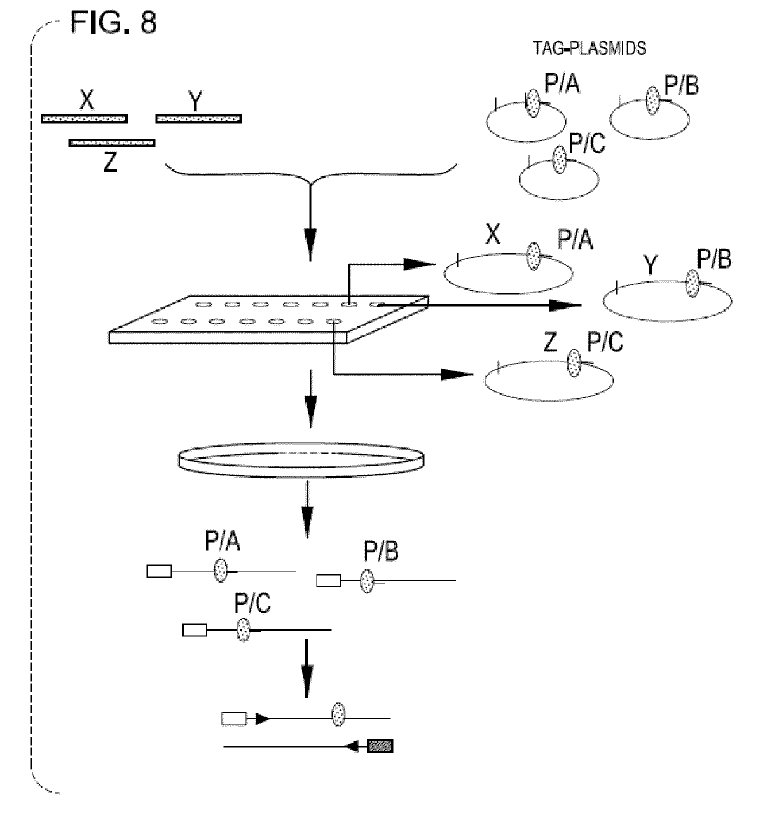

FIG. 8 is a flow chart for an alternative method disclosed herein. This method is described in detail in Example 6 below. In this embodiment, a vector library is constructed in which candidate DNA regulatory and/or promoter sequences are inserted into a plurality of vectors, each of which contains a different unique DNA TAG sequence and a probe sequence, such as a TaqMan® probe, that is common to each vector, and a reporter gene, such as the luciferase reporter gene. The TAG sequences are inserted into the individual vectors or plasmids, in between the luciferase reporter gene and the multiple cloning site into which the candidate sequence is to be inserted. Each vector construct, if it contains a functional transcriptional element, potentially drives the transcription of the probe sequence and the TAG sequence. Equal molar amounts of the vectors are pooled and transformed into reporter cells in a single tissue culture dish or tube, and cells grown for a desired amount of time. Total RNA is then extracted and purified in a single preparation, and the amount of RNA is determined using real time RT-PCR.

In one embodiment, the total RNA is then treated with calf intestinal phosphatase (CIP) and tobacco acid pyrophosphatase (TAP) and ligated to a RNA adaptor prior to real time RT-PCR analysis. The quantity of RNA generated from each plasmid is then determined by real time RT-PCR using a forward primer specific for the RNA adaptor ligated to the 5′ end of every intact RNA and reverse primers specific for the unique TAG sequences. The CIP/TAP treatment and RNA ligation step eliminates the problems of false amplification during PCR due to genomic DNA and plasmid DNA contamination in the RNA samples.

In certain embodiments, real time RT-PCR is performed using a one-step RT-PCR system (for example from Invitrogen) and a multi-tube plate, with each tube containing a PCR reaction master mix including a common forward primer, a unique universal labeled probe, and a different antisense TAG-specific primer in each well. A normalization probe and primer pairs can be included in each tube.

All the TAG sequences employed in the vectors have approximately the same melting temperature, which allows for the unbiased quantification of various mRNAs by RT-PCR under the same temperatures and ionic strength conditions. By mixing equal molar amounts of every DNA regulatory or promoter candidate and transforming the mixture into a single population of cells, a competitive environment is created for the various cis elements to recruit transcription factors. This closely mimics in vivo situations, eliminates the need for internal controls, reduces labor and costs, and enables researchers to simultaneously study large numbers of transcriptional element candidates.

The method enables the detection and quantification of mRNA levels, instead of reporter protein levels, and therefore should be unaffected by potentially interfering translation and posttranslational events unlike conventional reporter assays. The RT-PCR using a single TaqMan probe detection is simple and sensitive. In addition, the transcription initiation sites for new genes and for those less characterized genes can be readily determined by sequencing the RT-PCR product.

The design, operation and applications for the present disclosure will now be described in greater detail.

1. Design of a Library of DNA Tags that Will be Transcribed by the Putative DNA Promoter Sequences.

The TAG DNA sequences were DNA sequences composed of random nucleotides, that is each position had an equal probability of having any of the four deoxynucleotides (A, C, T, and G). Other bases, such as inosine, uracil, 5-methylcytosine, 8-azaguanine, 2,6-diaminopurine, 5 bromouracil, and other derivatives may be incorporated in their nucleotide form into the oligonucleotides. The length of the TAG sequence was short, preferably between about 16 by to about 200 bp, although a shorter or longer length may be used, but typically about 60 bp. Within a plurality of TAG sequences, each TAG sequence had approximately equivalent amounts of the nucleotides A, T, G, and C such that each TAG sequence had approximately the same melting temperature as the other TAGs, thereby allowing for the unbiased quantification of various mRNAs by hybridization under the same temperature and ionic strength conditions. Within a plurality of TAG sequences, the nucleotide sequence of each individual TAG sequence was unique amongst the plurality of TAGs. Each TAG did not share sequence homology with any other TAG used simultaneously in the same assay or with any DNA or RNA molecule that was labeled during the synthesis of the probe, regardless of the method used to synthesize the probe. A 60 by length of random nucleotides of the TAG sequence allowed for generation of a large number of unique TAGs that were highly unlikely to be found in nature. Additionally, the longer length of the TAG (e.g., about 60 bp) allowed for use of hybridization temperatures (e.g., 70° C.) that were high enough to prevent unspecific hybridization with partially homologous sequences. The GC content and thus melting temperature was normalized across the plurality of TAGs to ensure identical hybridization conditions for all of the TAG probes. To minimize cross-hybridization and for the highest specificity, all oligonucleotides were selected with a minimal length of sequence identity of no longer than six (6) bases. Low-complexity sequences with stretches of more than four (4) identical nucleotides were not allowed, thus avoiding difficulties in sequence similarity searching. Upon generation of the TAG sequences, the sequences were verified for the absence of homology amongst themselves. In some embodiments, the TAG sequences are examined against sequences deposited in public databases such as GenBank, EMBL, DDBJ and PDB using NCBI BLASTN to aid in determining if non-intended binding may occur. Oligonucleotides are generally synthesized as single strands by standard chemistry techniques, including automated synthesis. Many methods have been described for synthesizing oligonucleotides containing a randomized base. For example, a randomized position can be achieved by in-line mixing or using pre-mixed phosphoramidite precursors during an automated procedure (see, Ausbel et al., Current Protocols in Molecular Biology, Green Publishing, N.Y., 1995). Oligonucleotides are subsequently deprotected and may be purified by precipitation with ethanol, chromatographed using a size-exclusion or reversed-phase column, denaturing polyacrylamide gel electrophoresis, high-pressure liquid chromatography (HPLC), or other suitable method.

### 2. Construction of TAG-Plasmids

The TAG plasmids were derived from pTAG-basic (FIG. 7). This plasmid incorporates a pair of SfiI sites which generate two distinct 3 nucleotide-long nonsymmetrical sticky ends suitable for the directional insertion of the TAG oligonucleotides. The plasmid also incorporates a modified cDNA encoding firefly luciferase (luc+). This 1650 by cDNA was excised from the commercially available pGL3 using the restriction enzymes NcoI and XbaI. The wild-type coding region had been modified, in order to eliminate consensus sequences recognized by genetic regulatory proteins, thus helping to ensure that this reporter gene is unaffected by spurious host transcriptional signals. The plasmid also incorporates a 97 by long α-globin 3′UTR. The high level stability of α-globin mRNA, with a half-life from 24 to 60 hours, is attributed to a C-rich cis element in its 3′UTR, to which a protein complex binds to stabilize the mRNA. This protein complex is highly conserved from mouse to human and is found in a wide spectrum of tissues and cell lines. This sequence is sufficient to increase luciferase mRNA stability, with a half-life of 7 hours. In addition, the plasmid incorporates the SV40 polyA signal to efficiently polyadenylate the luciferase transcript, thus resulting in up to a five-fold increase of steady-state mRNA levels. The plasmid also incorporates a high copy number origin of replication from pUC19, but may alternatively contain a low copy number origin of replication, such as pBR322ColE1 ori/rop (15-20 copies per chromosome), pACYC177 p15A ori (10-12 copies per chromosome) or the CopyControl system (1, 10-50 copies per chromosome). Additionally, the plasmid includes the ampicillin and kanamycin resistance genes for selection of the pTAG derivatives in E. coli, the λ attP1 and attP2 sites for inserting promoter sequences by recombination using the Gateway® system, and a MCS for inserting promoter sequence candidates by DNA ligation. The MCS was present in two structurally different but functionally equivalent copies flanking the ccdB gene, a configuration that allows for using the ccdB gene as a selection marker for plasmids that incorporates promoter sequences, by recombination or by ligation. The CcdB protein targets DNA gyrase and inhibits its catalytic reactions. Cells taking up unreacted vectors with the ccdB gene will not grow. The plasmid also incorporates a short, synthetic polyA signal based on the highly efficient polyA signal of the rabbit β-globin gene. Placed upstream of the MCS, it will terminate spurious transcription, which may initiate within the vector backbone.

3. Insertion of DNA TAGs into pTAG-Basic

Typically, TAGs were obtained by annealing complementary 63 by oligonucleotides [(+)strand: (N)₆₀:ATA; (−)strand: (N)₆₀:GTG] that were then ligated into SfiI digested pTAG-basic. The ligation reaction was electroporated into a host strain, for example E. coli DB3.1, which contains a gyrase mutation (gyrA462) that renders it resistant to the ccdB. Because the sticky ends generated by both SfiI sites are incompatible, a very low background of self-circularized pTAG-basic vectors, or vectors with multiple TAGs in tandem, was generated. The presence of the TAGs in the various plasmids was verified by DNA sequencing. High-throughput production of TAGs followed a similar methodology. Synthesis of 63 by oligonucleotides was performed in two 96-well plates ((+) and (−) strands, respectively). The (+) and (−) strands were annealed in a 96-well plate, and ligated with SfiI digested, gel-purified pTAG basic. The ligation mixture was electroporated into electro-competent the E. coli DB3.1 host cells, using a 96-well electroporation plate. The bacterial clones were seeded into a 96-Deep-Well plate and the cultures were incubated for 18-24 hours at 37° C. at 250 rpm using a microtiter plate incubator shaker. Plasmid DNA purification was performed, either manually or via automation, for example using a BioRobot 3000 (Qiagen, Valencia, Calif.), and the presence of the TAGs verified via DNA sequencing (96-well format).

### 4. Preparation of Promoter Array Membranes

Oligonucleotide arrays were manufactured using nylon membranes. The (−) strand TAG oligonucleotides were synthesized in a 96-well plate format and resuspended in buffer, for example TE, pH 7.5, at a concentration of 100 μg/ml. Nylon membranes, for example Nytran™ SuPerCharge (Whatman PLC, Middlesex, UK), were cut (2 cm×4 cm) to fit 5.0 ml glass hybridization tubes. Oligonucleotides were either spotted manually in duplicate on the membranes (0.2 μl/spot) or oligonucleotide arrays printed using an array spotting robot, for example a Biorobotics MicroGrid (Genomic Solutions, Ann Arbor, Mich.). After spotting, the membranes were UV cross-linked twice using a Stratalinker 1800 at 120 mJ/sec, then baked at 70° C. for 1-2 hours. The printed membranes were sealed in parafilm and stored at −20° C. The quality of the membranes was validated by hybridizing 10% of the membranes with biotin-labeled (+) strand oligonucleotide TAGs. The 3′ end of the TAG oligonucleotides was labeled using terminal transferase and biotin-16-ddUTP. All TAGs were mixed together in equimolar amounts. The TAG mixture (100 μmol) was incubated in the presence of 1.0 nmol biotin-16-ddUTP and 50 U terminal transferase, following the manufacturer's recommendations. After a 15 minute incubation at 37° C., the end-labeled TAG probes were precipitated with LiCl, centrifuged and resuspended in ddH₂O. The labeling efficiency was checked by spotting a serial dilution of the labeling reaction and a standard on the nylon membrane. Detection was performed by chemiluminescence, for example with alkaline phosphatase-conjugated streptavidin, following the manufacturer's recommendations. Quantification was performed by densitometry. Upon validation of the quality of the biotin-labeled probes, the quality of the arrays was assessed by hybridizing the probes to the membranes using standard procedures, detecting them by chemiluminescence, and measuring the intensity of each spot by densitometry. The membranes were accepted upon observation of less than a variation of 5% of intensity and spot size.

### 5. Construction of Promoter-TAG Plasmids

Promoter sequence candidates were inserted into TAG plasmids using two methods. First, promoter sequence candidates were extracted from existing plasmids using endonucleases such as restriction enzymes and inserted into the pTAG plasmids, between sites located in the multiple cloning sites. Promoter sequence and pTAG plasmids were assembled by DNA ligation using standard protocols (see Crowe et al., Improved cloning efficiency of polymerase chain reaction (PCR) products after proteinase K digestion. Nucleic Acids Res. 1991 Jan. 11; 19(1):184); Ausubel, F. M., et al., Short Protocols in Molecular Biology). Alternatively, promoter sequences were amplified by PCR, using primers carrying attB1 and attB2 extensions, and using mammalian genomic DNA or other plasmids as templates. The PCR products were inserted into the pTAG plasmids using the Gateway® recombination system. A promoter sequence candidate may be provided by a computer-predicted model, DNA fragments from a collection of nucleotide sequences, such as a genomic library, deletion or site-directed mutants of a specific promoter, tissue-specific promoters, artificial promoters, etc. Clones containing the pTAG plasmids with the promoter inserts were cultured in LB medium in the presence of 50 μg/ml ampicillin or 25 μg/ml kanamycin. At various time points during cell growth, aliquots of each culture were taken, the cell density measured spectrophotometrically at 600 nm, and equal volumes of culture pooled. Plasmid DNA was extracted using an alkaline lysis method and purified using anion-exchange resin. In order to verify that all plasmids were present in the mixture in equimolar concentrations, the following manipulation was performed. All plasmids in the DNA mixture were linearized by restriction digestion, and separated on an agarose gel (0.7%). The resultant DNA fragments, with sizes ranging from 5 to 15 kb, were stained with ethidium bromide and quantitated by densitometry using a gel documentation system. The linearity of the assay was verified by quantifying serial dilutions of the plasmid restriction digestion.

### 6. Transfection and RNA Extraction

The purified plasmid DNA mixture containing equimolar amounts of the promoter plasmids was transfected into HL60, U937, and 293 cell lines. Per transfection, 1×10⁷ viable U937 cells were washed and resuspended in 0.4 ml RPMI medium. Plasmid DNA (20 μg) was added and the cell/DNA suspension was mixed gently by inversion. After a 5 minute incubation at 25° C., the cells were electroporated using a BTX ECM-600 electroporator with the following settings: 500 V capacitance and resistance, 950 μF capacitance, 186 ohms resistance, 200 V charging voltage. After the electroshock, the cells were transferred into a 10 cm diameter tissue culture dish containing 10 ml RPMI medium supplemented with 10% FBS. After 2 to 5 hours incubation at 37° C., cells were harvested by centrifugation at 10 krpm for 30 seconds. Cell pellets were lysed by addition of 300 μl TRIzol® reagent and total RNA was extracted according to the manufacturers protocol (Invitrogen, Carlsbad, Calif.) (see also Current Protocols in Molecular Biology, John Wiley & Sons). RNA was precipitated with isopropyl alcohol, resuspended in RNase-free TE, pH 7.5, and quantified by measuring the absorbance at 260 nm and 280 nm (ratio ˜2). RNA integrity was verified by agarose gel electrophoresis and ethidium bromide staining. The 28S and 18S rRNAs, represented in discrete individual bands, had a 2:1 intensity ratio. RNA samples with a visible degree of degradation were not further processed. In parallel, an equimolar mixture of promoter-less TAG plasmids were transfected and analyzed for mRNA expression using the array. This control detected the possible presence of cryptic promoter activity in the TAGs. The promoter-less TAG plasmids yielding above-background signals were discarded.

### 7. Labeling, Hybridization, and Detection

Radioactive cDNA probes were synthesized from total RNA. The total RNA was purified with TRIzol® (Invitrogen) and the concentration of the RNA was determined by the OD260 reading. One to five microgram of total RNA was mixed with MA5-a oligo (5′-TAGTCACTTCGATCGCTGAGG-3′; SEQ ID NO. 1), and the nucleotides dATP, dTTP, dGTG, and 32P-dCTP. The reaction was incubated at 80° C. for 3 minutes and then cooled to 42° C. Then added were 10× reverse transcription buffer (NEB), RNAse inhibitor, and M-MuLV reverse transcriptase (NEB). The reaction was mixed and incubated at 42° C. for 60 minutes, then denatured at 90° C. for 10 minutes.

The radioactive probes were hybridized to the membrane using Ultrahyb-oligo hybridization buffer (Ambion, Inc.) at 60° C. overnight. After washing the membrane twice with 2×SSC/1% SDS and twice with 1×SSC/1% SDS at 60° C., the bound probes were detected by autoradiography, using for example, Kodak Biomax Light Film (Carestream Health, Inc., New Haven, Conn.). The density of each spot was quantified with computer software, for example, Kodak 1D Image Analysis Software (Carestream Health, Inc., New Haven, Conn.).

In an alternate embodiment, biotin-labeled cDNA probes were synthesized from the total RNA. The probes were synthesized using the AmpoLabeling-LPR method developed by SuperArray Bioscience Corporation. This method increased the sensitivity of cDNA arrays by amplifying the cDNAs obtained by reverse transcription by up to 30 rounds of Linear Polymerase Replication (LPR). A 300 nucleotide long region from the 5′ end of the luciferase mRNAs, encompassing the 60 nucleotide TAGs, was reverse transcribed and amplified in the presence of biotin-labeled dUTP. The total RNA was annealed with primer complementary to the MA4 segment, in a thermal cycler at 70° C. for 3 minutes, cooled to 37° C. and incubated at 37° C. for 10 minutes. The annealed product was reverse transcribed using MMLV reverse transcriptase in presence of RNasin Ribonuclease Inhibitor. After inactivation of the reverse transcriptase and RNA hydrolysis at 85° C., the cDNAs were amplified by LPR with primer 5′-GGCTCGGCCTCTGAGCTAAT-3′ (SEQ ID NO. 2) located immediately upstream of the TAG, in the presence of biotin-16-dUTP, and a thermostable DNA-dependent DNA polymerase, using the following program: 85° C. for 5 minutes; then 30 cycles of 85° C. for 1 minute, 50° C. for 1 minute, 72° C. for 1 minute; followed by 72° C. for 5 minutes. The probe was then checked for biotin incorporation by making serial dilutions of the probe synthesis reaction, spotting 1 μl aliquots on a HyBond™ nylon membrane and detecting the probe using the ECL chemiluminescent detection kit. Probes that were detectable at 1000-fold dilutions or higher were used in the hybridizations.

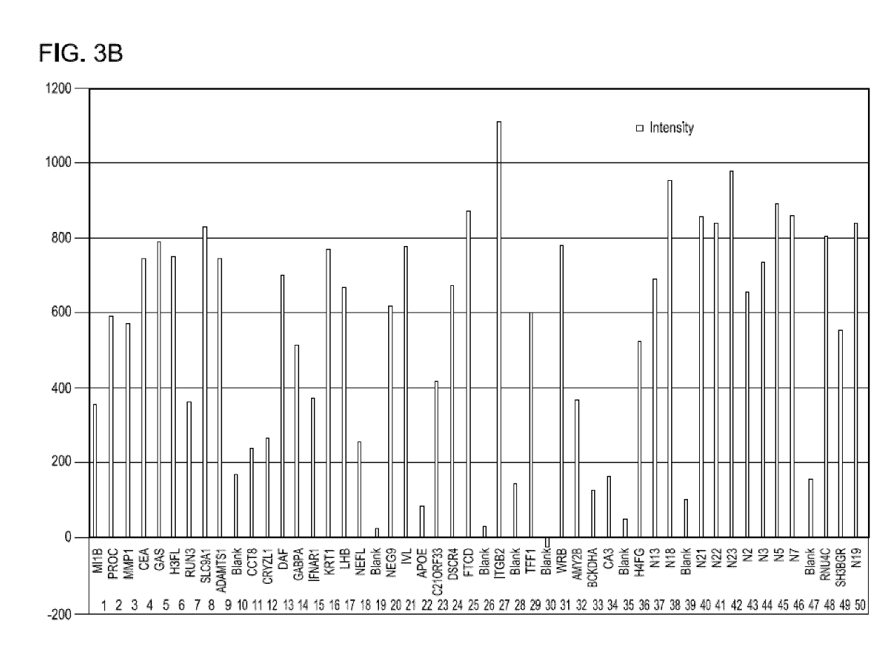

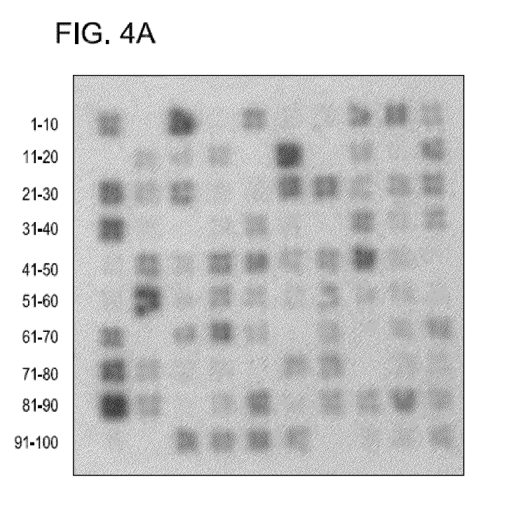

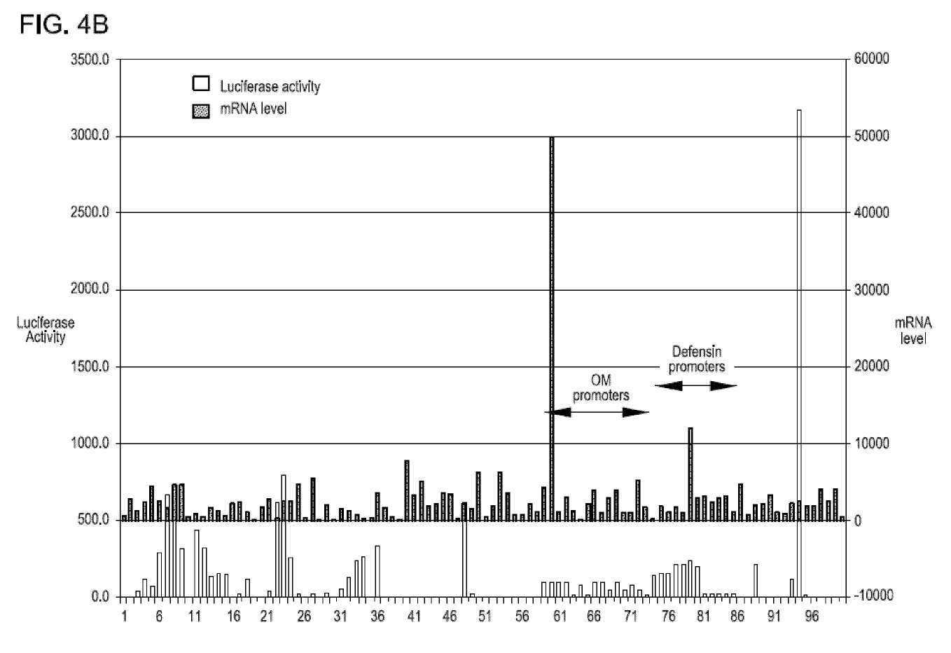

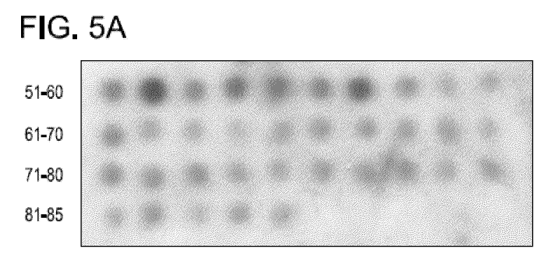

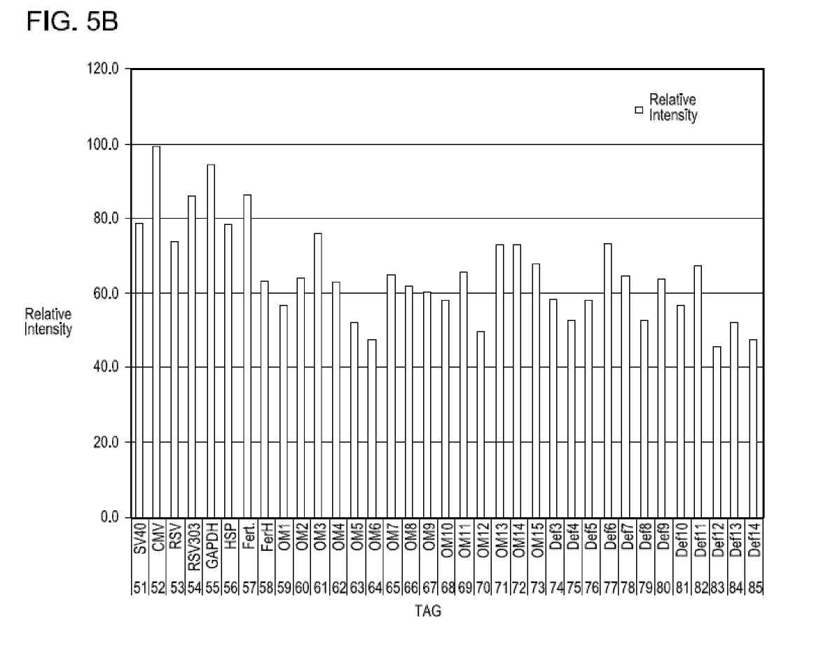

The hybridization of the biotinylated probes to the membranes was performed using the Ultrahyb-oligo hybridization buffer (Ambion Inc.), at 60° C. overnight. After washing the membrane twice with 2×SSC, 1% SDS and twice with 1×SSC, 1% SDS at 60C, the bound probes were detected by chemiluminescence using a streptavidin-alkaline phosphatase conjugate and following the manufacturer's protocol (CDP-Star Universal Detection Kit, Sigma). The image was acquired with a Kodak image station 440 for 1 hour (FIG. 3A, FIG. 4A, and FIG. 5A). The density from each spot was quantified using the Kodak 1D Image Analysis software.

The data presented in FIGS. 3A and 3B and FIGS. 4A and 4B show that: a) all the “blank” reporter-TAG plasmids which lack promoter sequences (# 10, 19, 26, 28, 30, 35, 39, and 47 in Table 1) give very low intensity signals, a fact, which suggests the absence of intrinsic promoter activity from the plasmid backbone; b) with the series of defensin promoters (#74-85), the clone expressing the highest mRNA level (# 79) is also the one expressing the highest level of luciferase. The data presented in FIGS. 5A and 5B show that: a) as expected, the viral CMV promoter appeared to be the strongest, a fact, which is well-documented in the scientific literature (U.S. Pat. Nos. 5,168,062 and 5,385,839; Cayer et al J Immunol Methods. 2007 Apr. 30; 322(1-2):118-27; Sakurai et al Gene Ther. 2005 October; 12(19):1424-33; Fabre et al. J Gene Med. 2006 May; 8(5):636-45); b) The GAPDH (glyceraldehyde-3-phosphate dehydrogenase) promoter was able to drive very high expression levels, which is consistent with observation made by others (Hirano T et al, Biosci Biotechnol Biochem. 1999; 63(7):1223-7; Punt P J et al. Gene. 1990; 93(1):101-9; Nagashima T et al., Biosci Biotechnol Biochem. 1994; 58(7):1292-6); c) the ferritin light-chain promoter was about 40% stronger than the Ferritin heavy chain promoter, a fact that supports findings made by Cairo et al. in rat liver (Biochem J. 1991; 275 (Pt 3):813-6); d) Promoters OM3 (TAG61) and Def6 (TAG77) produced the strongest hybridization signals in their respective groups (OM and Defensin promoters), a fact, which correlates with the luciferase activities determined previously (Ma et al., Nucleic Acids Res. 1999; 27(23):4649-57; Ma et al. J Biol. Chem. 1998 Apr. 10; 273(15):8727-40). Taken altogether, these data validate the present disclosure compared to other methods.

The following examples are offered by way of illustration, and not by way of limitation.

### EXAMPLES

### Example 1

### Construction of 100 pTAG-Reporter Plasmids

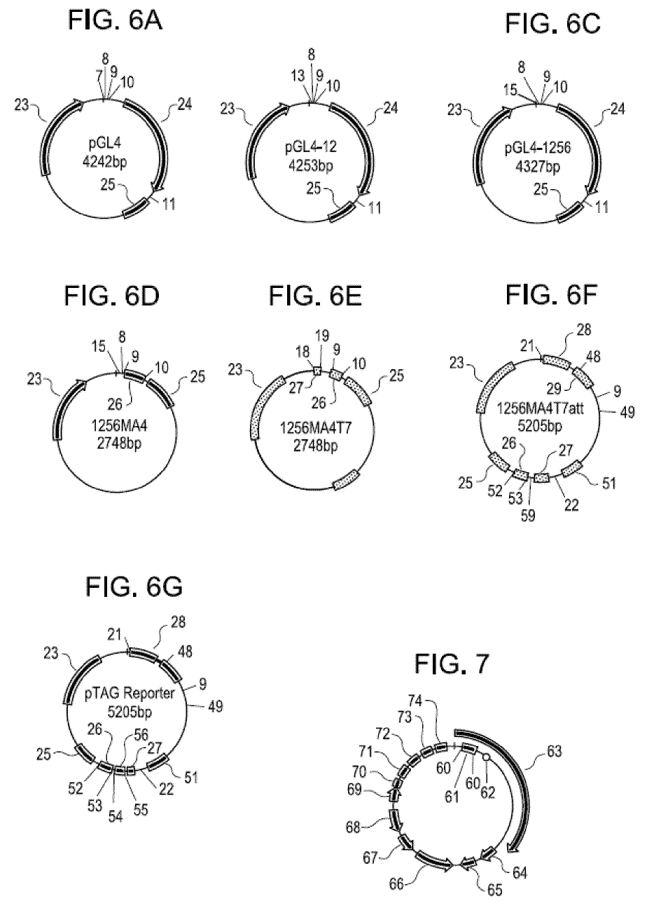

One hundred pTAG-plasmids featuring a multiple cloning site (MCS), attP sequences, a ccdB gene, a T7 promoter, a unique 60 bp-long reporter TAG, a specific MA4 segment, a 3-frame translation stop codon, a hemoglobin RNA stabilization fragment and a poly-A signal were constructed. The construction was performed in 6 steps (FIG. 6). First, a partial MCS was inserted, between the SfiI sites of plasmid pGL4 (Promega, Madison, Wis.). All the cloning sites from the original pGL4 plasmid were deleted and replaced with EcoRI, KpnI, SacI, NheI, XhoI, BgIII sites, and followed by two sets of SfiI/BgII sites separated by a CG dinucleotide. The two sets of SfiI sites allowed for the directional insertion of TAG sequences. The dinucleotide CG between the SfiI sites created a unique restriction site (SmaI/XmaI), which revealed useful to facilitate plasmid digestion with SfiI, either by insertion of a ˜170 bp-long spacer fragment to dissociate both SfiI sites, or by digestion of the plasmid sequentially with SmaI and then SfiI.

In the second step, a second partial MCS was inserted between the XhoI and BglII sites of pGL4-12. The resulting plasmid (pGL-1256) contained BglII, ApaI, NruI, KpnI, XhoI SacI, BglII, NheI, EcoRV, and MluI sites following the existing MCS. As a result, pGL-1256 contained two structurally different but functionally equivalent MCS surrounding the ApaI and NruI sites, a feature useful for cloning promoter sequence candidates in the TAG-plasmids.

In the third step, the sequence encoding the luciferase reporter gene (NcoI-XbaI fragment) was replaced with an 80-mer oligonucleotide which contained a specific 25 bp-long sequence (MA4), a three-frame translation stop codon, and a RNA stabilization sequence derived from human alpha globin gene. The MA4 facilitated the synthesis of TAG-specific probes from mRNAs.

In the fourth step, the resulting plasmid 1256MA4 was digested with EcoRV and MluI, which allowed for insertion of an oligonucleotide that contained the bacteriophage T7 RNA polymerase promoter sequence. The presence of the T7 promoter allowed for synthesis of biotinylated RNA probes by in vitro transcription, a method which increased the sensitivity of the assay by at least one order of magnitude.

In the fifth step, the Gateway® sequences attP-ccdB-chloramphenicol-resistance gene were amplified by PCR using plasmid pDONR-201 as template (Invitrogen Inc., Carlsbad, Calif.) and the following primers: sense-tcgggccccaaataatgattttattttgactgatag (SEQ ID NO. 3) and antisense-atgggcccaaataatgattttattttgactgatagtgacctgttc (SEQ ID NO. 4). The PCR product was inserted into the ApaI site of plasmid 1256MA4T7, generating plasmid 1256MA4T7att.

Finally, plasmid 1256MA4T7att was digested with BglI and 60 bp-long ds oligonucleotides (TAG) were directionally inserted into the plasmid. In total, we created 100 reporter plasmids—pTAG-Reporter 1 to 100. These plasmids were used to generate the 92 promoter-TAG plasmids. The remaining 8 pTAG-Reporter plasmids were used as blank.

These 100 pTAG-Reporter plasmids are used for cloning putative promoters into the MCS, using either conventional methods (restriction digestion and ligation), or the Gateway® technology with attB-modified PCR products.

### Example 2

### Manual and Robotic Production of Macro-Array Membranes

First, three nylon membranes: BrightStar®-Plus (Ambion Inc., Austin, Tex.), Tropilon-Plus™ (Applied Biosystems, Foster City, Calif.), and Nytran™ SuperCharge (Whatman PLC, Middlesex, UK) were compared for their ability in being printed with short oligonucleotides. The 63 bp-long oligonucleotides complementary to the TAGs present on the TAG-reporter plasmids were manually spotted on the membranes, and hybridized with the biotin end-labeled sense TAG oligonucleotides. BrightStar®-Plus (Ambion Inc., Austin, Tex.) was selected for use in subsequent experiments as this membrane produced the best results in terms of low background, sharpness of the signal spots, and the observation the rough surface of the BrightStar®-Plus membrane produced stronger signals than the smooth surfaces of the other two membranes, without increasing the background. The nylon membranes were cut (2×4 cm) to fit 5-mL glass hybridization tubes and the 8-well hybridization plates (SuperArray Inc., Frederick, Md.).

Next, the amount of oligonucleotides to be spotted on the membrane was optimized. Stock solutions for all the reverse strand TAG oligonucleotides were made by reconstituting the lyophilized products in TE pH 7.5 to 100 μM. Serial dilutions of 20×, 60×, 180×, 540× and 1620× were made. Using a 2 μL Pipetman, the diluted oligonucleotides (0.2 μl) were spotted manually, in duplicate, on the membrane. Following hybridization of the membrane with biotin end-labeled sense-strand TAG oligonucleotide probes, detection of the signals was performed by chemiluminescence using the Southern-Star kit (Applied Biosystems, Foster City, Calif.). The 20-fold dilutions produced a strong and clean signal spots, and were selected.

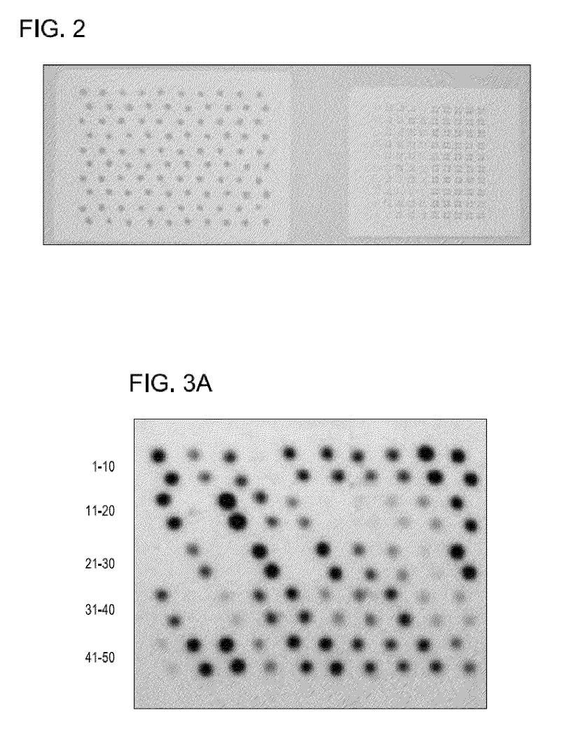

The same diluted oligonucleotides (n=100) (FIG. 2) were printed using a Biorobotics MicroGrid array spotting robot (Genomic Solutions, Ann Arbor, Mich.) at the microarray facility of the University of Idaho Environmental Biotechnology Institute (Moscow, ID). Each oligonucleotide was printed as a quadruple spot. Both types of membranes were air-dried at room temperature for 10 min and then UV-crosslinked twice using a Stratalinker 1800 (Stratagene) at 120 mJ/sec, then baked at 70° C. for 2 hours. The printed membranes were then sealed in parafilm and stored at 4° C. The size of the membrane was designed to fit into convenient small containers such as 2-mL microcentrifuge tubes and 8-well plates.

### Example 3

### Cloning of 92 Human and Viral Promoter Sequences into the TAG-Reporter Plasmids

Ninety-two human and viral promoter sequences (TABLE 1) were cloned into the TAG-reporter plasmids using the Gateway® system. They included 12 defensin promoters and 15 Oncostatin M promoters, 57 genomic DNA fragments from both EPD and chromosome 21, which have been studied experimentally for promoter activity, and 8 well-known promoters (SV40, CMV, wild-type and mutant RSV, GAPDH, HSP, FerL, and FerH).

First, the promoter sequences were amplified by PCR, using human chromosomal DNA or plasmids as templates, and primers carrying attB sequence extensions. The PCR products were inserted into the pTAG-reporter plasmids in place of the ccdB and chloramphenicol-resistance genes by in vitro recombination using the BP clonase (Invitrogen, Carlsbad, Calif.). The recombinant plasmids were introduced into E. coli Top10 using the heat-shock procedure, and amplified. Recombinant clones lacking promoter inserts were obtained at a frequency of about 1:200. To ascertain the correct clones, the plasmid DNAs of each clone were prepared and analyzed by agarose gel electrophoresis separately. Plasmid DNAs were quantified by spectrophotometry. Finally, equimolar amounts were pooled at a final concentration of 0.4 μg DNA/μL.

In the context of screening plasmid libraries of putative promoters, E. coli clones are arrayed in 96-well plates. The bacteria (not their plasmid DNA) are pooled and amplified in the same flask. Their plasmid DNA is purified in a single preparation, before being transfected into the same cell population.

### Example 4

### Testing the Promoter Detective Method with 92 Promoter-TAG Plasmids

The method was performed with the 92 promoter-TAG and 8 blank reporter-TAG plasmids. Different amounts (4, 16, 64 μg) of equimolar mixtures of these plasmids were transfected into HEK 293 cells using Lipofectamine™ 2000 (Invitrogen, Carlsbad, Calif.). After 14 and 25 hours culture at 37° C., cells were harvested. Total RNA was extracted and purified using the TRIzol®-based method (Invitrogen, Carlsbad, Calif.). Biotin labeled cDNA probes were synthesized from the total RNA. The probes were synthesized using the AmpoLabeling LPR method (SuperArray Bioscience Corp., Frederick, Md.). The sensitivity of cDNA arrays was increased by amplifying the cDNAs obtained by reverse transcription by up to 30 rounds of Linear Polymerase Replication (LPR). A 300 nucleotide long region, encompassing the 60 nucleotide TAGs, was reversed transcribed and amplified in the presence of biotin labeled dUTP. The 2.5 μg total RNA was annealed with primer complementary to the MA4 segment, in a thermal cycler at 70° C. for 3 minutes, cooled to 37° C. and incubated at 37° C. for 10 minutes. The annealed product was reverse transcribed using MMLV reverse transcriptase and RNA hydrolysis at 85° C., the cDNAs were amplified by LPR with primer 5′-GGCTCGGCCTCT GAGCTAAT-3′ (SEQ ID NO. 2) located immediately upstream of the TAG, in the presence of biotin 16 dUTP, and a thermostable DNA dependent DNA polymerase, with the following program: 85° C. for 5 minutes; then 30 cycles of 85° C. for 1 minute; 50° C. for 1 minute; 72° C. for 1 minute; followed by 72° C. for 5 minutes. The probe was then checked for biotin incorporation by making serial dilutions of the probe synthesis, spotting 1 μl aliquots onto a HyBond™ nylon membrane (Amersham, Little Chalfont, UK) and detecting the probe using the ECL chemiluminescent detection kit. Probes detectable at 1000-fold dilutions or higher were used in the hybridizations.

The hybridization of the biotinylated probes to the membranes was performed using the Ultrahyb-oligo hybridization buffer (Ambion Inc.), at 60° C. overnight. After washing the membrane twice with 2×SSC, 1% SDS and twice with 1×SSC, 1% SDS at 60C, we detected bound probes by chemiluminescence using a streptavidin-alkaline phosphatase conjugate and following the manufacturer's protocol (CDP-Star Universal Detection Kit, Sigma). The image was acquired with a Kodak image station 440 for 1 hour (FIG. 4A). The density from each quadruple spot was quantified using the Kodak 1D Image Analysis software. The results indicate: a) all the “blank” reporter-TAG plasmids which lack promoter sequences (# 10, 19, 26, 28, 30, 35, 39, and 47 in Table 1) give very low intensity signals, a fact, which suggests the absence of intrinsic promoter activity from the plasmid backbone; b) with the series of defensin promoters (#74-85), the clone expressing the highest mRNA level (# 79) is also the one expressing the highest level of luciferase.

### Example 5

### Testing the Promoter Detection Method with 35 Promoter-TAG Plasmids

The method was tested with a set of 35 promoter-TAG plasmids. Twenty μg of an equimolar mixture of these plasmids were transfected into U937 cells by electroporation. After 7 hours culture at 37° C., cells were harvested. Total RNA was extracted and purified using the TRIzol®-based method (Invitrogen. Carlsbad, Calif.), and quantified by spectrophotometry (Abs260 nm).

Radioactive cDNA probes were synthesized as follows. One microgram total RNA in 6.3 μL H₂O was mixed with 0.7 μL of 100 μM MA5-a oligonucleotide (5′-TAGTCACTTCGAT CGCTGAGG-3′; SEQ ID NO. 1), 1.1 μL of 5 mM each of dATP/dTTP/dGTG, and 1.9 μL ³²P dCTP. The reaction mixture was heated to 80° C. for 3 minutes and then cooled down to 42° C. Then 1.5 μL 10× reverse transcription buffer (New England Biolabs), 0.75 μL RNAse inhibitor, and M-MuLV reverse transcriptase (New England Biolabs) were added, and the reaction was performed at 42° C. for 60 minutes. The probes were then denatured at 90° C. for 10 minutes.

The hybridization of the radioactive probes to the membranes was performed using the Ultrahyb-oligo hybridization buffer (Ambion Inc.), at 60° C. overnight. After washing the membrane twice with 2×SSC, 1% SDS and twice with 1×SSC, 1% SDS at 60° C., bound probes were detected by autoradiography using a Kodak Biomax Light film. The density of each spot was quantified using the Kodak 1D Image Analysis software (FIGS. 5A and 5B) where the autoradiogram was obtained by hybridizing radioactive TAG-cDNA probes to a membrane spotted with complementary TAG strands. The intensities of the various spots were compared, relative to the signal obtained with the CMV promoter. As expected, the viral CMV promoter appeared to be the strongest, a fact, which is well-documented in the scientific literature (U.S. Pat. Nos. 5,168,062 and 5,385,839; Cayer et al J Immunol Methods. 2007 Apr. 30; 322(1-2):118-27; Sakurai et al Gene Ther. 2005 October; 12(19):1424-33; Fabre et al. J Gene Med. 2006 May; 8(5):636-45). The GAPDH (glyceraldehyde-3-phosphate dehydrogenase) promoter was able to drive very high expression levels, which is consistent with observation made by others (Hirano T et al, Biosci Biotechnol Biochem. 1999; 63(7):1223-7; Punt P J et al. Gene. 1990; 93(1):101-9; Nagashima T et al., Biosci Biotechnol Biochem. 1994; 58(7):1292-6). Also, the ferritin light-chain promoter was about 40% stronger than the Ferritin heavy chain promoter, a fact that supports findings made by Cairo et al. in rat liver (Biochem J. 1991; 275 (Pt 3):813-6). Promoters OM3 (TAG61) and Def6 (TAG77) produced the strongest hybridization signals in their respective groups (OM and Defensin promoters), a fact, which correlates with the luciferase activities determined previously (Ma et al., Nucleic Acids Res. 1999; 27(23):4649-57; Ma et al. J Biol. Chem. 1998 Apr. 10; 273(15):8727-40). Taken altogether, these data validate the present disclosure compared to other methods.

### Example 6

### mRNA Quantification Using Real Time RT-PCR

a. Plasmid Construction

Promega's pGL4.10 plasmid was used as the backbone for plasmid construction, mainly because it contains the luciferase reporter gene and because of its low background transcription. The basic plasmid contained a 46 bp-long fragment inserted immediately 3′ of HindIII site. This 46 bp-long fragment was composed of two parts: the first 25 base pairs was an artificially designed probe sequence (5′-FAM-TCGTCAGTCGCAGTCTCAGACTCAC-BHQ-3′; SEQ ID NO: 5) common to every plasmid, with 56% GC, and with Tₘ 69° C. The second part was a TAG sequence of 21 base pairs. Nine such plasmids were constructed with each having a unique computer program-generated 21 bp-long TAG sequence. The original multiple cloning site with unique SfiI sites from pGL4.10 remained upstream of the TAG for cloning candidate transcriptional regulatory elements.

b. Transfection of the TAG Plasmids into a Reporter Cell Line

1) HEK293 cells from ATCC (The American Type Culture Collection) were used as the host for the transfection. The HEK 293 cells were grown in DMEM/FBS (10%) supplemented with penicillin/streptomycin to approximately 75% confluence. 24 hours before the transfection, the cells were detached from the tissue culture dish using trypsin/EDTA, washed with PBS, and resuspended in DMEM/FBS supplemented with penicillin/streptomycin at a concentration of 100,000 viable cells/mL. 0.5 mL of the diluted cell suspension were seeded into each well of a 24-well plate. Immediately before the transfection, the medium was replaced with 0.5 mL fresh DMEM/FBS devoid of penicillin/streptomycin and the ˜75% confluence of the cell culture was checked under the microscope.

2) The plasmids were purified individually and pooled in equimolar ratio. The pooled plasmids were transfected into a single dish of HEK 293 cells using Lipofectamine™ (Invitrogen), with about 400 ng pooled plasmids for every 10,0000 cells, from which total RNA was purified.

3) The detailed procedure for Lipofectamine™ transfection is as follows: per well, 200 ng pooled plasmid mixture were resuspended in 50 μL Opti-MEM® (Invitrogen, Carlsbad, Calif.), and vortexed briefly. In a separate tube 1 μL Lipofectamine™ (Invitrogen) was mixed with 50 μL Opti-MEM®. After 5 minutes incubation at room temperature, both tubes were mixed to each other and incubated for 20 more minutes at room temperature to allow the formation of a complex DNA-liposome. The above ˜100 μL mixture was added to each well of the 24-well plate and mixed gently by rocking the plate back and forth.

4) To purify total RNA, 300 μL TRIzol® (Invitrogen, Carlsbad, Calif.) were added to each well. The mixture was pipetted up and down several times to break the cells. The lysate was transferred into 1.5 mL microcentrifuge tubes for immediate experimental procedures or stored at −80° C.

c. Measurement of Candidate DNA Activity

1) The concentration of the RNA samples was measured with a spectrophotometer and the quality and the integrity of the RNA was checked on a denaturing agarose gel. The 28S ribosomal RNA band was twice the intensity of the 18S band and both bands were sharp on the gel with no smear, which indicated the absence of RNA degradation. 7 μL total RNA (about half of the RNA samples that were prepared in section b above) were treated with calf intestinal phosphatase (CIP) to remove the free 5′-phosphates from other molecules present in the sample, such as ribosomal RNA, fragmented mRNA, tRNA and contaminating genomic and plasmid DNA. The cap structure found on intact 5′ ends of mRNA was not affected by CIP, but this cap was removed by treatment with tobacco acid pyrophosphatase (TAP), leaving a 5′-monophosphate. The CIP- and TAP-treated total RNA samples were ligated to a 25 by RNA adaptor 5′-GCUGAUGCGAUGAAUGAACACGAAA-3′ (SEQ ID NO: 6) using a T4 RNA ligase. This RNA adaptor cannot ligate to dephosphorylated RNA because these molecules lack the 5′-phosphate necessary for ligation.

2) The CIP/TAP-treated RNA sample was used in a real-time RT-PCR assay to determine the quantities of each RNA that are derived from the plasmids, using an Applied Biosystems 7500 PCR machine. Invitrogen's One-Step Quantitative RT-PCR System was used for the detection and quantification of mRNA. This system combines the SuperScript III Reverse Transcriptase (RT) that can synthesize cDNA at a temperature range of 42-60° C., an optimized random primer mix for first strand cDNA synthesis, and a Platinum Taq DNA Polymerase whose polymerase activity is blocked by an antibody at ambient temperatures until the denaturation step in PCR cycling. Both cDNA synthesis and PCR were performed in a single tube. The CIP/TAP-treated RNA adaptor-ligated total RNA was used as template with a common forward primer matching the RNA adaptor sequence and reverse primers matching the TAG sequences. A FAM™ dye-labeled TaqMan oligonucleotide (Biosearch Technologies, Novato, Calif.) was used as probe in the real-time RT PCR with the following settings: 50° C. for 30 minutes for reverse transcription followed by 40 cycles of 95° C. for 15 seconds and 55° C. for 30 seconds. Fluorescence was measured at each 55° C. step.

TABLE 1

| | Gene | Promoter | Refseq or |
|---|---|---|---|
| TAG # | symbol | size (bp) | Accession # |
| 1 | MT1B | 471 | M13484 |
| 2 | PROC | 495 | NM_000312 |
| 3 | MMP1 | 477 | NM_002421 |
| 4 | CEA | 508 | NM_002483 |
| 5 | GAS | 539 | NM_000805 |
| 6 | H3FL | 506 | NM_003537 |
| 7 | RUN3 | 356 | K00777 |
| 8 | SLC9A1 | 509 | XM_046881 |
| 9 | ADAMTS1 | 560 | NM_006988 |
| 10 | Blank | | |
| 11 | CCT8 | 528 | NM_006585 |
| 12 | CRYZL1 | 583 | NM_005111 |
| 13 | DAF | 557 | NM_000574 |
| 14 | GABPA | 611 | NM_002040 |
| 15 | IFNAR1 | 667 | NM_000629 |
| 16 | KRT1 | 520 | NM_006121 |
| 17 | LHB | 494 | NM_000894 |
| 18 | NEFL | 495 | NM_006158 |
| 19 | Blank | | |
| 20 | NEG9 | 407 | N/A |
| 21 | IVL | 500 | NM_005547 |
| 22 | APOE | 509 | NM_000041 |
| 23 | C21ORF33 | 689 | NM_004649 |
| 24 | DSCR4 | 688 | NM_005867 |
| 25 | FTCD | 596 | NM_006657 |
| 26 | Blank | | |
| 27 | ITGB2 | 647 | NM_000211 |
| 28 | Blank | | |
| 29 | TFF1 | 605 | NM_003225 |
| 30 | Blank | | |
| 31 | WRB | 639 | NM_004627 |
| 32 | AMY2B | 488 | NM_020978 |
| 33 | BCKDHA | 481 | NM_000709 |
| 34 | CA3 | 518 | NM_005181 |
| 35 | Blank | | |
| 36 | H4FG | 222 | NM_003542 |
| 37 | NEG13 | 376 | N/A |
| 38 | NEG18 | 503 | N/A |
| 39 | Blank | | |
| 40 | NEG21 | 444 | N/A |
| 41 | NEG22 | 418 | N/A |
| 42 | NEG23 | 259 | N/A |
| 43 | NEG2 | 285 | N/A |
| 44 | NEG3 | 460 | N/A |
| 45 | NEG5 | 488 | N/A |
| 46 | NEG7 | 466 | N/A |
| 47 | Blank | | |
| 48 | RNU4C | 305 | M15957 |
| 49 | SH3BGR | 588 | NM_007341 |
| 50 | NEG19 | 483 | N/A |
| 51 | SV | 330 | N/A |
| 52 | CMV | 655 | N/A |
| 53 | RSV | 396 | N/A |
| 54 | RSV303 | 396 | N/A |
| 55 | GAPDH | 532 | N/A |
| 56 | HSP | 464 | N/A |
| 57 | FerL | 270 | N/A |
| 58 | FerH | 180 | N/A |
| 59 | OM1 (pGL3BomB1) | 189 | BC011589 |
| 60 | OM2 (N1) | 304 | BC011589 |
| 61 | OM3 (3STAT) | 300 | BC011589 |
| 62 | OM4 (3STATm) | 300 | BC011589 |
| 63 | OM5 (3STATmm) | 300 | BC011589 |
| 64 | OM6 (N1 ApI) | 304 | BC011589 |
| 65 | OM7 (N1 SpI mutation) | 304 | BC011589 |
| 66 | OM8 (N1 3STATmm) | 304 | BC011589 |
| 67 | OM9 (RI) | 194 | BC011589 |
| 68 | OM10 (StuI) | 94 | BC011589 |
| 69 | OM11 (2STATm) | 194 | BC011589 |
| 70 | OM12 (N1 2STATmm) | 304 | BC011589 |
| 71 | OM13 (1STAT) | 109 | BC011589 |
| 72 | OM14 (1STATm) | 109 | BC011589 |
| 73 | OM15 (TATA) | 31 | BC011589 |
| 74 | Def3 (B/3) | 619 | AA321199 |
| 75 | Def4 (AvaI) | 497 | AA321199 |
| 76 | Def5 (HincII) | 321 | AA321199 |
| 77 | Def6 (HinfI) | 299 | AA321199 |
| 78 | Def7 (ApoI) | 203 | AA321199 |
| 79 | Def8 (Sau96I (7)) | 164 | AA321199 |
| 80 | Def9 (ScrfI (9)) | 144 | AA321199 |
| 81 | Def10 (ScrfI (TATA)) | 144 | AA321199 |
| 82 | Def11 (Tru9I) | 111 | AA321199 |
| 83 | Def12 (Tru9ITATA) | 111 | AA321199 |
| 84 | Def13 (Tru9ITATAm) | 111 | AA321199 |
| 85 | Def14 (Tru9ITATAm2) | 111 | AA321199 |
| 86 | ALB | 517 | NM_000477 |
| 87 | NEG11 | 468 | N/A |
| 88 | HLCS | 645 | NM_000411 |
| 89 | NEG12 | 522 | N/A |
| 90 | NEG1 | 500 | N/A |
| 91 | NEG6 | 480 | N/A |
| 92 | ORM1 | 499 | NM_000607 |
| 93 | PKNOX1 | 593 | NM_004571 |
| 94 | USP16 | 581 | NM_006447 |
| 95 | IGSF5 | 622 | AF121782 |
| 96 | NEG10 | 406 | N/A |
| 97 | NEG16 | 202 | N/A |
| 98 | NEG17 | 339 | N/A |
| 99 | PCP4 | 625 | NM_006198 |
| 100 | TCRD | 333 | M21624 |

We claim:

1. A method for detecting DNA regulatory sequences or promoter sequences comprising:
(a) inserting each of a plurality of DNA regulatory sequence candidates or promoter sequence candidates into one of a plurality of vectors wherein each of the vectors comprises a unique TAG sequence and wherein each DNA regulatory sequence candidate or promoter sequence candidate is inserted in a position to drive transcription of the TAG sequence;
(b) inserting each of the vectors into one of a plurality of cloning host cells;
(c) growing the cloning host cells to the same optical density and pooling the cloning host cells;
(d) extracting and purifying the vectors from the cloning host cells and inserting the vectors into a reporter cell line or a nuclear extract thereof; and
(e) extracting mRNA from the reporter cell line or the nuclear extract thereof and analyzing the mRNA,
wherein the presence of mRNA corresponding to the TAG sequence from a specific vector is indicative of the presence of a DNA regulatory sequence or promoter sequence in the vector.

2. The method of claim 1, wherein the vector is a plasmid.

3. The method of claim 1, wherein the TAG sequence is between about 16 base pairs to about 200 base pairs in length.

4. The method of claim 1, wherein about equal amounts of the purified vectors are transferred into the reporter cell line.

5. The method of claim 1, wherein each of the plurality of vectors further comprises one or more multiple-cloning sites, and a transcription termination signal.

6. The method of claim 5, wherein the transcription termination signal is a poly-A signal.

7. The method of claim 5, wherein the TAG sequence is located 3′ to the DNA regulatory sequence candidate or promoter sequence candidate and 5′ to the transcription termination site.

8. The method of claim 5, wherein each of the vectors further comprises at least one component selected from the group consisting of: a DNA recombination sequence; a negative selection marker; a RNA polymerase promoter sequence; a translation stop codon; and a RNA stabilization fragment.

9. The method of claim 1, wherein the mRNA extracted from the reporter cell lines is directly labeled or is used as a template for cDNA or probe synthesis and the labeled mRNA, cDNA or probe is analyzed using an array that comprises sequences that are identical or complementary to the TAG sequences.

10. The method of claim 9, wherein the cDNA or probe contains a label.

11. The method of claim 9, wherein the mRNA is directly labeled.

12. The method of claim 9, wherein the mRNA is analyzed with an array, wherein the array comprises complementary sequences to the TAG sequences, and wherein the complementary sequences are antisense strands.

13. The method of claim 9, wherein the cDNA is analyzed with an array, wherein the array comprises complementary sequences to the cDNA of the TAG sequences, and wherein the complementary sequences are sense strands.

14. The method of claim 9, wherein the labeled mRNA, cDNA or probe hybridizes to the array and the label of the mRNA, cDNA or probe has a detectable response.

15. A method for detecting DNA regulatory sequences or promoter sequences comprising:
(a) inserting each of a plurality of DNA regulatory sequence candidates or promoter sequence candidates into one of a plurality of vectors wherein each of the vectors comprises a unique TAG sequence and wherein each DNA regulatory sequence candidate or promoter sequence candidate is inserted in a position to drive transcription of the TAG sequence;
(b) inserting each of the vectors into one of a plurality of cloning host cells;
(c) growing the cloning host cells to the same optical density;
(d) extracting and purifying the vectors from the cloning host cells;
(e) pooling the purified vectors and inserting the vectors into a reporter cell line; and
(f) extracting mRNA from the reporter cell line and analyzing the mRNA,
wherein the presence of mRNA corresponding to the TAG sequence from a specific vector is indicative of the presence of a DNA regulatory sequence or promoter sequence in the vector.

16. A method for detecting DNA regulatory sequences or promoter sequences comprising:
(a) inserting each of a plurality of DNA regulatory sequence candidates or promoter sequence candidates into one of a plurality of vectors wherein each of the vectors comprises a unique TAG sequence, a reporter gene and a labeled probe sequence, wherein each vector contains the same probe sequence and wherein each DNA regulatory sequence candidate or promoter sequence candidate is inserted in a position to drive transcription of the TAG sequence;
(b) pooling equimolar amounts of the vectors and transfecting the vectors into reporter cells;
(c) growing the reporter cells;
(d) extracting and purifying RNA from the reporter cells; and
(e) quantifying the amount of mRNA generated from each vector using real time reverse transcription polymerase chain reaction (real time RT-PCR),
wherein the presence of mRNA corresponding to the TAG sequence from a specific vector is indicative of the presence of a DNA regulatory sequence or promoter sequence in the vector.

17. The method of claim 16, wherein a RNA adaptor is ligated to the 5′ end of intact mRNA after purification of the RNA, and wherein the real time RT-PCR employs a forward primer that is specific for the sequence of the RNA adaptor and reverse primers that are specific for the unique TAG sequences.

18. A plurality of vectors into which a plurality of DNA regulatory sequence candidates or DNA promoter sequence candidates can be inserted wherein each of the vectors comprises a unique TAG sequence, one or more multiple-cloning sites and a transcription termination signal.

19. The plurality of vectors of claim 18, wherein each of the vectors further comprises at least one component selected from the group consisting of: a DNA recombination sequence; a negative selection marker; a RNA polymerase promoter sequence; a translation stop codon; and a RNA stabilization fragment.

20. A kit comprising a plurality of vectors according to claim 18 and an array, wherein the array comprises sequences that are identical to or complementary to the TAG sequences.
